(19) United States
(12) Patent Application Publication
(10) Pub. No.: US 2021/0162039 A1
(43) Pub. Date: Jun. 3, 2021

(54) MULTIEPITOPE FUSION ANTIGENS FOR VACCINATION AND METHODS OF MAKING AND USING SUCH ANTIGENS

(71) Applicant: Kansas State University Research Foundation, Manhattan, KS (US)

(72) Inventors: Weiping Zhang, Manhattan, KS (US); Ying Fang, Manhattan, KS (US)

(21) Appl. No.: 17/250,020

(22) PCT Filed: May 13, 2019
(86) PCT No.: PCT/US2019/032029

§ 371 Date: Nov. 4, 2020

Related U.S. Application Data
(60) Provisional application No. 62/670,126, filed on May 11, 2018.

(51) Int. Cl. A61K 39/225; A61K 39/108; C07K 14/165

(52) U.S. Cl. CPC A61K 39/225; A61K 2039/523; C07K 14/165; A61K 39/0258

(57) ABSTRACT

The present disclosure provides vaccines or immunogenic compositions against clinical signs, symptoms, and losses attributable to or caused by infection with PEDV and/or ETEC. Antigenic epitopes from PEDV and ETEC are used to construct an immunogenic composition, preferably in the form of a multi-epitope fusion antigen or as a live strain through E. coli.

### STATEMENT REGARDING FEDERALLY SPONSORED RESEARCH & DEVELOPMENT

This invention was made with government support under contract no. 2017-67015-26632 awarded by the United States Department of Agriculture. The government has certain rights in the invention.

### SEQUENCE LISTING

The present application includes a sequence listing submitted with this application through EFS-Web. This sequence listing is incorporated by reference herein in its entirety.

### BACKGROUND OF THE INVENTION

Porcine epidemic disease virus (PEDV) and enterotoxigenic Escherichia coli (ETEC) are emerging threats for U.S. swine producers. A safe and protective vaccine would be the most effective way to protect against PEDV and ETEC. Safety, however, is a concern for the modified live virus (MLV), which might be reverted to a virulent strain and subsequently shed the reverted viruses.

PED (Porcine Epidemic Diarrhea) is caused by a coronavirus named Porcine Epidemic Disease virus (PEDV). Two different types of PEDV are recognized: Type I only affects growing pigs whereas Type II affects all ages including suckling pigs and mature sows. Type I is seen as a low level endemic disease in the UK with pro-active surveillance only detecting very low numbers annually. Type II is a more virulent strain that caused the death of over 1 million pigs in the USA in 2013-14, with up to 100% mortality seen in piglets less than 7 days old.

PEDV damages the villi (cell surface) in the pig gut, reducing the amount of absorptive surface area which results in a loss of fluid, diarrhea and dehydration. Up to 100% of sows in a herd may be affected, showing mild to watery diarrhea after the PEDV enters a herd, but a strong immunity develops over two to three weeks and the colostrum then protects the piglets. The virus usually disappears spontaneously from small intensive breeding herds however in larger outdoor breeding herds not all the pigs may become infected the first time round and there may be recrudescence of the disease over a longer time frame.

The clinical signs of PED are very age-specific and much more severe in younger animals. In very young piglets there is profuse, watery diarrhea, without blood or mucus, which is usually yellow-green in color, often accompanied with vomiting and anorexia which may lead to death in up to 100% of the piglets less than a week old. Pigs over a week of age typically recover but with growth rate losses of circa 10%. When older animals (nursery, grower, finisher, sows, boars) become infected they may go off feed for two to four days, have loose feces or watery diarrhea with no blood or mucus and vomit—dehydration is common. A death rate of 1 to 3% in the post-weaned animals is typical.

The incubation period of PEDV is typically two to four days and the first clinical signs in a herd are seen approximately four days after PEDV enters the herd. When the virus is first introduced on to the farm there is a rapid spread of diarrhea across all breeding (Type II) and growing pigs (Type I and II) with almost 100% morbidity (pigs affected) within 5 to 10 days, although in outdoor extensively kept pigs this time may be protracted.

In breeding adults there may be associated production losses such as; increase in returns to service if sows/gilts are affected in early pregnancy; Agalactia (loss of milk production) in farrowed sows; reduced libido in boars; and abortions.

Escherichia coli is a gram negative peritrichously flagellated bacteria belonging to the family Enterobatteriaceae and is the causative agent of a wide range of diseases in pigs, including neonatal diarrhea and post-weaning diarrhea (PWD), which are important causes of death occurring worldwide in suckling and weaned pigs respectively.

Two main pathotypes are involved in enteric colibacillosis: enterotoxigenic E.coli (ETEC) and enteropathogenic E. coli (EPEC). ETEC is the most important pathotype in swine and includes different pathotypes (this term is used to describe strains characterized by different combinations of toxins and fimbriae). Outbreaks of neonatal and post-weaning diarrhea due to ETEC infection, generally affecting a high proportion of pigs, are often recurrent in the same herds and require expensive control measures. Enteric colibacillosis may result in significant economic losses due to mortality, decreased weight gain, cost for treatments, vaccinations and feed supplements. ETEC possess fimbriae which adhere to enterocytes and elaborate one or several enterotoxins that induce secretory diarrhea, causing some of the most significant diseases in the pig industry worldwide, such as neonatal colibacillosis and PWD.

Infection by ETEC is usually signaled by the appearance of diarrhea. Piglets from gilts may be more severely affected than piglets nursing sows. The severity of the diarrhea varies. The hypersecretory diarrhea usually has an alkaline pH but varies in color. It may be clear and watery, especially in neonates, but may be white or yellow, influenced by type of ingesta and duration of the disease. Sick pigs occasionally vomit but vomiting is not as prominent as with transmissible gastroenteritis (TGE). As diarrhea continues, there is progressive dehydration and the hair coat becomes roughened. Body temperature often is subnormal. Shivering often is noted unless an adequate supplementary heat source, such as heat lamps, is available. Signs are similar in pigs of various ages but tend to be more severe in younger pigs. Death losses can be severe if husbandry and environmental conditions are poor. Diarrhea tends to persist until intervention is accomplished. ETEC is one of the most common causes of neonatal septicemia and polyserositis. Often, strains associated with septicemia are not enteropathogenic. Further clinical signs include lesions and dehydration is the most obvious clinical sign. The small intestine and colon may contain excess watery fluid or may be distended and gas-filled. There may be mild reddening and congestion of the stomach. Lesions often are surprisingly mild, especially in very young piglets. However, in outbreaks caused by certain pathogenic strains, usually in older pos-tweaned pigs, there can be marked congestion of the gastrointestinal tract. Microscopy of the mucosa of the small intestine reveals many coliforms adhered to microvilli of intestinal epithelial cells. Villi usually are intact. With some strains of ETEC, there may be necrosis of some villi and microvascular thrombosis in the lamina propria. ETEC is a common cause of septicemia in neonates. In those cases, there is fibrinous polyserositis and arthritis.

At necropsy, pigs infected with ETEC may exhibit no observable lesions other than those associated with dehydration. However, some may exhibit mild to moderate intestinal hyperemia. Histologic examination of the small intestine reveals bacilli adherent multifocally or diffusely on the villous surface. Gram staining of impression smears from the ileum of most clinically infected pigs reveals large numbers of gram-negative bacilli, typically with few other organisms present and large numbers of E. coli can be cultured from those tissues. ETEC cultured from infected neonatal pigs may be either hemolytic or non-hemolytic, but only hemolytic ETEC colonize weaned pigs.

### BRIEF DESCRIPTION OF THE DISCLOSURE

In general, this technology applies to the creation of safe but effective vaccines or immunogenic compositions against clinical signs, symptoms, and losses attributable to or caused by infection with PEDV and/or ETEC.

In one aspect of the present disclosure, five epitopes were identified from the PEDV spike protein using web-based computer software, and these PEDV epitopes were embedded into a monomeric heat-labile (LT) toxoid or a holotoxin-structured LT for LT-PEDV MEFA (multi-epitope fusion antigen), and each LT-PEDV MEFA was expressed in E. coli as a recombinant protein or as a live strain expressing the holotoxin-structured antigen. The recombinant monomeric LT-PEDV protein can be used as a subunit vaccine to immunize pregnant sows and to produce maternal antibodies to protect born piglets against PEDV, or to immunize the mothers and also piglets with the live E. coli strain expressing the MEFA as a live vaccine strain for both passive-acquired and active-acquired immunity against PEDV. In some forms of the present disclosure, at least one, two, three, four, or all five of the epitopes are embedded in the monomeric heat-labile (LT) toxoid or a holotoxin-structured LT for LT-PEDV MEFA (multi-epitope fusion antigen), and each LT-PEDV MEFA was expressed in E. col as a recombinant protein or as a live strain expressing the holotoxin-structured antigen. In other forms, a sequence encoding a protein having at least 80%, more preferably 85%, and still more preferably at least 90, 91, 92, 93, 94, 95, 96, 97, 98, 99, 99.5, or 100% sequence homology is embedded in the monomeric heat-labile (LT) toxoid or a holotoxin-structured LT for LT-PEDV MEFA (multi-epitope fusion antigen), and each LT-PEDV MEFA was expressed in E. coli as a recombinant protein or as a live strain expressing the holotoxin-structured antigen. In some forms, the epitope expresses a polypeptide comprising a sequence having at least 80%, more preferably 85%, and still more preferably at least 90, 91, 92, 93, 94, 95, 96, 97, 98, 99, 99.5, or 100% sequence homology with any one of SEQ ID NOS. 1-5.

In another aspect of the present disclosure, the monomeric LT-PEDV can be used as the antigen for a PEDV subunit vaccine development, and the holotoxin-structured LT-PEDV MEFA expressed in a live E. coli or other bacteria strains can be a PEDV live vaccine strain. In either instance, at least one, two, three, four, or all five of the epitopes are embedded in the monomeric heat-labile (LT) toxoid or a holotoxin-structured LT for LT-PEDV MEFA (multi-epitope fusion antigen), and each LT-PEDV MEFA was expressed in E. coli as a recombinant protein or as a live strain expressing the holotoxin-structured antigen. In other forms, a sequence encoding a protein having at least 80%, more preferably 85%, and still more preferably at least 90, 91, 92, 93, 94, 95, 96, 97, 98, 99, 99.5, or 100% sequence homology is embedded in the monomeric heat-labile (LT) toxoid or a holotoxin-structured LT for LT-PEDV MEFA (multi-epitope fusion antigen), and each LT-PEDV MEFA was expressed in E. coli as a recombinant protein or as a live strain expressing the holotoxin-structured antigen. In some forms, the epitope expresses a polypeptide comprising a sequence having at least 80%, more preferably 85%, and still more preferably at least 90, 91, 92, 93, 94, 95, 96, 97, 98, 99, 99.5, or 100% sequence homology with any one of SEQ ID NOS. 1-5.

Because the monomeric LT-PEDV MEFA or the holotoxin-structured LT-PEDV include up to 5 epitopes from the PEDV S protein, and no live virus is involved in the vaccine product, both products are safe. Advantageously, this live E. coli strain is a nonpathogenic porcine E. coli field strain, not a live PEDV virus, and thus, it will not reverse to virulence and is safe. As with the other forms at least one, two, three, four, or all five of the epitopes are embedded in the monomeric heat-labile (LT) toxoid or a holotoxin-structured LT for LT-PEDV MEFA (multi-epitope fusion antigen), and each LT-PEDV MEFA was expressed in E. coli as a recombinant protein or as a live strain expressing the holotoxin-structured antigen. In other forms, a sequence encoding a protein having at least 80%, more preferably 85%, and still more preferably at least 90, 91, 92, 93, 94, 95, 96, 97, 98, 99, 99.5, or 100% sequence homology is embedded in the monomeric heat-labile (LT) toxoid or a holotoxin-structured LT for LT-PEDV MEFA (multi-epitope fusion antigen), and each LT-PEDV MEFA was expressed in E. coli as a recombinant protein or as a live strain expressing the holotoxin-structured antigen. In some forms, the epitope expresses a polypeptide comprising a sequence having at least 80%, more preferably 85%, and still more preferably at least 90, 91, 92, 93, 94, 95, 96, 97, 98, 99, 99.5, or 100% sequence homology with any one of SEQ ID NOS. 1-5.

Advantageously, the holotoxin-structured LT-PEDV binds GM receptors at intestinal epithelium, thus this LT-PEDV acts as a broad antigen and also a self-adjuvant to enhance induction of local mucosal antigen specific immunity.

In another aspect of the present disclosure, safe and effective vaccines and immunogenic compositions against ETEC have also been developed. In some forms, the vaccines comprise at least one ETEC epitope or a sequence encoding a protein having at least 80%, more preferably 85%, and still more preferably at least 90, 91, 92, 93, 94, 95, 96, 97, 98, 99, 99.5, or 100% sequence homology with any one of SEQ ID NOS. 9-35. In some forms, these epitopes are combined with or fused with a carrier or backbone to carry and present each epitope. In some forms, the carrier is a carrier protein. In some forms, the carrier protein is a modified chicken ovalbumin gene or E. coli fimbrial subunit genes.

In another aspect of the present disclosure, a composition comprising a combination of antigenic ETEC epitopes is provided. In some forms, the epitopes are continuous B cell epitopes. In some forms, the epitopes are selected from LT_{A} subunit epitopes, K88 FaeG subunit epitopes, F18 adhesin minor subunit epitopes, and any combination thereof. In some forms, the composition comprises at least one epitope from at least two different continuous B cell epitopes selected from LT_{A} subunit epitopes, K88 FaeG subunit epitopes, and F18 adhesin minor subunit epitopes. In some forms, the composition comprises at least one epitope from each of the following continuous B cell epitopes: LT_{A} subunit epitopes, K88 FaeG subunit epitopes, and F18 adhesin minor subunit epitopes. In some forms, the LT_{A} subunit epitopes encode a protein having at least 80%, more preferably 85%, and still more preferably at least 90, 91, 92, 93, 94, 95, 96, 97, 98, 99, 99.5, or 100% sequence homology with any one of SEQ ID NOS. 9-19. In some forms, the K88 FaeG subunit epitopes encode a protein having at least 80%, more preferably 85%, and still more preferably at least 90, 91, 92, 93, 94, 95, 96, 97, 98, 99, 99.5, or 100% sequence homology with any one of SEQ ID NOS. 20-28. In some forms, the F18 adhesin minor subunit epitopes encode a protein having at least 80%, more preferably 85%, and still more preferably at least 90, 91, 92, 93, 94, 95, 96, 97, 98, 99, 99.5, or 100% sequence homology with any one of SEQ ID NOS. 29-35.

In another aspect of the present disclosure, an immunogenic composition as described above is administered to an animal in need thereof. In some forms, the animal is a mammal, preferably a pig. In some forms, the administration result in a reduction in the incidence of and/or the severity of clinical signs and/or symptoms of infection by PEDV and/or ETEC. Preferably, the reduction in incidence and/or severity is at least 5%, more preferably 10, 15, 20, 25, 30, 35, 40, 45, 50, 55, 60, 65, 70, 75, 80, 85, 90, 95, or even 100% in comparison to an animal that has not received an administration of the immunogenic composition described herein.

In another aspect of the present disclosure, an immunogenic composition as described above is combined with at least one further component selected from the group consisting of an adjuvant, a pharmaceutical-acceptable carrier; a protectant; an immunomodulatory agent, a pharmaceutical acceptable salt, and any combination thereof.

### BRIEF DESCRIPTION OF THE DRAWINGS

FIG. 1 is presents four panels of the LT-PEDV chimeric gene construction and fusion protein detection. Panel A is an illustration of the LT-PEDV chimeric gene structure wherein the PEDV S1 and S2 B-cell epitopes and inserted locations were marked; Panel B is a photograph of Coomassie blue staining showing protein purity of the LT-PEDV fusion protein; Panel C is a photograph demonstrating the detection of the LT-PEDV fusion protein with anti-LT rabbit serum; and Panel D is a photograph demonstrating detection of the LT-PEDV fusion protein with serum from a neonatal pig infected with PEDV;

FIG. 2 is a schematic illustration of the construction and detection of the holotoxin-structured LT-PEDV MEFA. The first 153 amino acids of the native LT A1 peptide were replaced with the same length peptide from the monomeric LT-PEDV MEFA to construct the LT-PEDV MEFA to be expressed in a holotoxin structure. The holotoxin-structured LT-PEDV MEFA, the LT A subunit, and LT B subunit were detected with anti-CT antibodies;

FIG. 3 is a graph illustrating the expressed LT-PEDV MEFA induced IgG antibodies to PEDV spike protein including the S1 and S2 domains;

FIG. 4 is a series of photographs illustrating that antibodies in mice serum immunized with the LT-PEDV MEFA neutralize PEDV infection at concentrations ranging from 1:16 to 1:256;

FIG. 5 is a set of photographs illustrating the LT_{A} epitope-ovalbumin fusion construction, expression and antigenicity wherein the top panel is a diagram to show the epitope-albumin structure, the middle panel uses Coomassie blue staining to show fusion protein expression and extraction, and the bottom panel is a Western blot with anti-LT antibodies and wherein e1 to e11 represent epitope-ovalbumin fusions with each LT epitope;

FIG. 6A is set of photographs wherein the top panel illustrates Coomassie blue staining showing K88 FaeG epitope-ovalbumin fusion expression and extraction and the bottom panel is a Western blot with anti-LT antibodies to detect fusion with e1, e2, e3, e5, or e9;

FIG. 6B is a graph illustrating an ELISA demonstrating that each fusion protein was recognized with anti-FaeG antiserum;

FIG. 7A is set of photographs wherein the top panel illustrates Coomassie blue staining showing F18 FedF epitope-ovalbumin fusion expression and extraction and the bottom panel is a Western blot illustrating that the epitope fusion proteins were recognized with anti-F18 antiserum;

FIG. 7B is a graph illustrating an ELISA demonstrating that each fusion protein was recognized with anti-F18 antiserum;

FIG. 8 is a graph illustrating mouse serum anti-LT IgG titers (log₁₀) from the group subcutaneously immunized with epitope-ovalbumin fusion protein (e1 to e11) or the control group immunized with protein carrier ovalbumin (−). Bars indicated the mean titer in each group. E1 to e11 represented groups of mice immunized with fusion proteins carrying different LT_{A1} epitopes. Each dot referred to an IgG titer from each mouse (solid dots for mice immunized with epitope-ovalbumin fusion protein empty data for control mice);

FIG. 9 is a graph illustrating mouse serum anti-K88 IgG titers (log₁₀) from the group subcutaneously immunized with FaeG epitope-CfaB fusion protein (e1 to e9). Bars indicated the mean titer in each group. K88e1 to K88e9 represented groups of mice immunized with fusion proteins carrying different K88 FaeG epitope 1 to 9. Each dot referred to an IgG titer from each individual mouse;

FIG. 10 is a graph illustrating mouse serum anti-F18 IgG titers (log₁₀) from the group subcutaneously immunized with FedF epitope-CfaB fusion protein (e1 to e7). Bars indicated the mean titer in each group. F18e1 to F18e7 represented groups of mice immunized with fusion proteins carrying different FedF epitopes. Each dot refers to an IgG titer from an individual mouse;

FIG. 11 is a graph illustrating mouse serum antibody in vitro neutralization activity against LT enterotoxicity. Mouse serum samples from the group immunized with each epitope-ovalbumin fusion protein (e1 to e11), the control group immunized with ovalbumin protein (ovalbumin), or the group immunized with PBS (−) mixed with 20 ng LT were incubated with T-84 cells. T-84 intracellular cGMP levels were measured with EIA cAMP kit (Enzo Life Science). LT (20 ng) was directly added to T-84 cells to show enterotoxicity in stimulation of cAMP levels in T-84 cells, and T-84 cells without LT or serum (medium) to show baseline cAMP levels;

FIG. 12 is a graph illustrating mouse serum antibody in vitro adherence inhibition against K88 fimbrial ETEC strain 30302 (K88/LT/STa/STb). Mouse serum samples from the group immunized with each epitope-ovalbumin fusion protein (K88e1 to K88e9), the control group were incubated to ETEC bacteria 3030-2. Each serum and bacteria mixture was added to IPEC-2 cells culture in 24-well plates. After 1 h culture in CO₂ incubator at 37° C., non-adherent bacteria were washed off. Bacteria adhered to IPEC-2 cells were collected, serial diluted, and plated on LB plates. After overnight growth, bacteria CFUs were counted; and

FIG. 13 is a graph illustrating Mouse serum antibody in vitro adherence inhibition against K88 fimbrial ETEC strain 8516. Mouse serum samples from the group immunized with each epitope-ovalbumin fusion protein (F18e1 to F18e7) and the control group were incubated with F18 fimbrial bacteria 8516. Each serum and bacteria mixture was added to IPEC-2 cells culture in 24-well plates. After culturing for 1 hour in a CO₂ incubator at 37° C., non-adherent bacteria were washed off. Bacteria adhered to IPEC-2 cells were collected, serial diluted, and plated onto LB plates. After overnight growth, bacteria CFUs were counted.

### DETAILED DESCRIPTION OF THE INVENTION

The terms “vaccine”, “immunological composition”, and “immunogenic composition”, as used herein, are used interchangeably and refer to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

“Adjuvant” as used herein, refers to a substance that helps and/or enhances the effect of a drug, treatment, or biologic system. Adjuvants can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). John Wiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, Jun. 1996). Persons skilled in the art can also refer to U. S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the composition can include one or more pharmaceutical-acceptable or veterinary-acceptable carriers. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like.

A “protectant” as used herein, refers to an anti-microbiological active agent, such as for example Gentamycin, Merthiolate, and the like. In particular adding a protectant is most preferred for the preparation of a multi-dose composition. Those anti-microbiological active agents are added in concentrations effective to prevent the composition of interest from any microbiological contamination or for inhibition of any microbiological growth within the composition of interest.

The methods and compositions of the present disclosure can also comprise the addition of any “stabilizing agent”, which refers to a component used to increase and/or maintain product shelf-life and/or to enhance stability. Some examples of stabilizing agents include saccharides, trehalose, mannitol, saccharose and the like.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

Those of skill in the art will understand that the composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others. Suitable adjuvants, are those described above.

According to a further aspect, the immunogenic composition of the present disclosure further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

The immunogenic compositions described herein can further include one or more other immunomodulatory agents such as, e. g., interleukins, interferons, or other cytokines. The immunogenic compositions can also include Gentamicin and Merthiolate. In another preferred embodiment, the present disclosure contemplates vaccine compositions comprising from about 1 μg/ml to about 60 μ/ml of antibiotics, and more preferably less than about 30 μg/ml of antibiotics.

The terms “immunogenic protein”, “immunogenic polypeptide” or “immunogenic amino acid sequence” as used herein refer to any amino acid sequence which elicits an immune response in a host against a pathogen comprising said immunogenic protein, immunogenic polypeptide or immunogenic amino acid sequence. An “immunogenic protein”, “immunogenic polypeptide” or “immunogenic amino acid sequence” as used herein, includes the full-length sequence of any proteins, analogs thereof, or immunogenic fragments thereof. By “immunogenic fragment” is meant a fragment of a protein which includes one or more epitopes and thus elicits the immunological response against the relevant pathogen. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, June 28-Jul. 3, 1998.

An “immunological active component” as used herein means a component that induces or stimulates the immune response in an animal to which said component is administered. According to a preferred embodiment, said immune response is directed to said component or to a microorganism comprising said component. According to a further preferred embodiment, the immunological active component is an attenuated microorganism, including modified live virus (MLV), a killed-microorganism or at least an immunological active part of a microorganism.

“Immunological active part of a microorganism” as used herein means a protein-, sugar-, and or glycoprotein containing fraction of a microorganism that comprises at least one antigen that induces or stimulates the immune response in an animal to which said component is administered. According to a preferred embodiment, said immune response is directed to said immunological active part of a microorganism or to a microorganism comprising said immunological active part.

In the present description, the terms polypeptide, peptide and protein are interchangeable.

Polypeptide fragment according to the disclosure is understood as designating a polypeptide containing at least 5 consecutive amino acids, preferably 10 consecutive amino acids or 15 consecutive amino acids.

In the present disclosure, specific polypeptide fragment is understood as designating the consecutive polypeptide fragment encoded by a specific fragment nucleotide sequence according to the disclosure.

Homologous polypeptide will be understood as designating the polypeptides having, with respect to the natural polypeptide, certain modifications such as, in particular, a deletion, addition or substitution of at least one amino acid, a truncation, a prolongation, a chimeric fusion, and/or a mutation. Among the homologous polypeptides, those are preferred whose amino acid sequence has at least 80%, preferably 90, 91, 92, 93, 94, 95, 96, 97, 98, and 99% homology with the sequences of amino acids of polypeptides according to the disclosure.

Specific homologous polypeptide will be understood as designating the homologous polypeptides such as defined above and having a specific fragment of polypeptide according to the disclosure.

In the case of a substitution, one or more consecutive or nonconsecutive amino acids are replaced by “equivalent” amino acids. The expression “equivalent” amino acid is directed here at designating any amino acid capable of being substituted by one of the amino acids of the base structure without, however, essentially modifying the biological activities of the corresponding peptides and such that they will be defined by the following. These equivalent amino acids can be determined either by depending on their structural homology with the amino acids which they substitute, or on results of comparative tests of biological activity between the different polypeptides, which are capable of being carried out. By way of example, the possibilities of substitutions capable of being carried out without resulting in an extensive modification of the biological activity of the corresponding modified polypeptides will be mentioned, the replacement, for example, of leucine by valine or isoleucine, of aspartic acid by glutamic acid, of glutamine by asparagine, of arginine by lysine etc., the reverse substitutions naturally being envisageable under the same conditions.

The specific homologous polypeptides likewise correspond to polypeptides encoded by the specific homologous nucleotide sequences such as defined above and thus comprise in the present definition the polypeptides which are mutated or correspond to variants which can exist in PEDV, and which especially correspond to truncations, substitutions, deletions and/or additions of at least one amino acid residue.

Specific biologically active fragment of a polypeptide according to the disclosure will be understood in particular as designating a specific polypeptide fragment, such as defined above, having at least one of the characteristics of polypeptides according to the disclosure, especially in that it is: capable of inducing an immunogenic reaction directed against a PEDV and/or ETEC; and/or capable of being recognized by a specific antibody of a polypeptide according to the disclosure; and/or capable of linking to a polypeptide or to a nucleotide sequence of PEDV and/or ETEC; and/or capable of exerting a physiological activity, even partial, such as, for example, a dissemination or structural activity; and/or capable of modulating, of inducing or of inhibiting the expression of a PEDV and/or ETEC gene or one of its variants, and/or capable of modulating the replication cycle of PEDV and/or ETEC in the cell and/or the host organism.

The polypeptide fragments according to the disclosure can correspond to isolated or purified fragments naturally present in a PEDV and/or ETEC or correspond to fragments which can be obtained by cleavage of said polypeptide by a proteolytic enzyme, such as trypsin or chymotrypsin or collagenase, or by a chemical reagent, such as cyanogen bromide (CNBr) or alternatively by placing said polypeptide in a very acidic environment, for example at pH 2.5. Such polypeptide fragments can likewise just as easily be prepared by chemical synthesis, from hosts transformed by an expression vector according to the disclosure containing a nucleic acid allowing the expression of said fragments, placed under the control of appropriate regulation and/or expression elements.

“Modified polypeptide” of a polypeptide according to the disclosure is understood as designating a polypeptide obtained by genetic recombination or by chemical synthesis as will be described below, having at least one modification with respect to the normal sequence. These modifications will especially be able to bear on amino acids at the origin of a specificity, of pathogenicity and/or of virulence, or at the origin of the structural conformation, and of the capacity of membrane insertion of the polypeptide according to the disclosure. It will thus be possible to create polypeptides of equivalent, increased or decreased activity, and of equivalent, narrower, or wider specificity. Among the modified polypeptides, it is necessary to mention the polypeptides in which up to 5 amino acids can be modified, truncated at the N- or C-terminal end, or even deleted or added.

As is indicated, the modifications of the polypeptide will especially have as objective: to render it capable of modulating, of inhibiting or of inducing the expression of PEDV and/or ETEC gene and/or capable of modulating the replication cycle of PEDV and/or ETEC in the cell and/or the host organism, of allowing its incorporation into vaccine compositions, and/or of modifying its bioavailability as a compound for therapeutic use.

The methods allowing said modulations on eukaryotic or prokaryotic cells to be demonstrated are well known to the person skilled in the art. It is likewise well understood that it will be possible to use the nucleotide sequences coding for said modified polypeptides for said modulations, for example through vectors according to the disclosure and described below, in order, for example, to prevent or to treat the pathologies linked to the infection.

The preceding modified polypeptides can be obtained by using combinatorial chemistry, in which it is possible to systematically vary parts of the polypeptide before testing them on models, cell cultures or microorganisms for example, to select the compounds which are most active or have the properties sought.

Chemical synthesis likewise has the advantage of being able to use unnatural amino acids, or nonpeptide bonds. Thus, in order to improve the duration of life of the polypeptides according to the disclosure, it may be of interest to use unnatural amino acids, for example in D form, or else amino acid analogs, especially sulfur-containing forms, for example.

Finally; it will be possible to integrate the structure of the polypeptides according to the disclosure, its specific or modified homologous forms, into chemical structures of polypeptide type or others. Thus, it may be of interest to provide at the N- and C-terminal ends compounds not recognized by the proteases.

The nucleotide sequences coding for a polypeptide according to the disclosure are likewise part of the disclosure.

The disclosure likewise relates to nucleotide sequences utilizable as a primer or probe, characterized in that said sequences are selected from the nucleotide sequences according to the disclosure.

The cloning and the sequencing of the PEDV and/or ETEC genome has allowed it to be identified, after comparative analysis with the nucleotide sequences of other porcine circoviruses, that, among the sequences of fragments of these nucleic acids, were those which are strictly specific to the PEDV and/or ETEC and those which correspond to a consensus sequence of a different pathogen other than the PEDV and/or ETEC.

It will be possible to use said consensus nucleotide sequences, said corresponding polypeptides as well as said antibodies directed against said polypeptides in procedures or sets for detection and/or identification such as described below, in place of or in addition to nucleotide sequences, polypeptides or antibodies according to the disclosure, specific to PEDV and/or ETEC.

The disclosure additionally relates to the use of a nucleotide sequence according to the disclosure as a primer or probe for the detection and/or the amplification of nucleic acid sequences.

The nucleotide sequences according to the disclosure can thus be used to amplify nucleotide sequences, especially by the PCR technique (polymerase chain reaction) (Erlich, 1989; Innis et al., 1990; Rolfs et al., 1991; and White et al., 1997). These oligodeoxyribonucleotide or oligoribonucleotide primers advantageously have a length of at least 8 nucleotides, preferably of at least 12 nucleotides, and even more preferentially at least 20 nucleotides. Other amplification techniques of the target nucleic acid can be advantageously employed as alternatives to PCR.

The nucleotide sequences of the disclosure, in particular the primers according to the disclosure, can likewise be employed in other procedures of amplification of a target nucleic acid, such as: the TAS technique (Transcription-based Amplification System), described by Kwoh et al. in 1989; the 3SR technique (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

The polynucleotides of the disclosure can also be employed in techniques of amplification or of modification of the nucleic acid serving as a probe, such as: the LCR technique (Ligase Chain Reaction), described by Landegren et al. in 1988 and improved by Barany et al. in 1991, which employs a thermostable ligase; the RCR technique (Repair Chain Reaction), described by Segev in 1992; the CPR technique (Cycling Probe Reaction), described by Duck et al. in 1990; the amplification technique with Q-beta replicase, described by Miele et al. in 1983 and especially improved by Chu et al. in 1986, Lizardi et al. in 1988, then by Burg et al. as well as by Stone et al. in 1996.

In the case where the target polynucleotide to be detected is possibly an RNA, for example an MRNA, it will be possible to use, prior to the employment of an amplification reaction with the aid of at least one primer according to the disclosure or to the employment of a detection procedure with the aid of at least one probe of the disclosure, an enzyme of reverse transcriptase type in order to obtain a cDNA from the RNA contained in the biological sample. The cDNA obtained will thus serve as a target for the primer(s) or the probe(s) employed in the amplification or detection procedure according to the disclosure.

The detection probe will be chosen in such a manner that it hybridizes with the target sequence or the amplicon generated from the target sequence. By way of sequence, such a probe will advantageously have a sequence of at least 12 nucleotides, in particular of at least 20 nucleotides, and preferably of at least 100 nucleotides.

The disclosure also comprises the nucleotide sequences utilizable as a probe or primer according to the disclosure, characterized in that they are labeled with a radioactive compound or with a nonradioactive compound. The unlabeled nucleotide sequences can be used directly as probes or primers, although the sequences are generally labeled with a radioactive element (32P, 35S, 3H, 125I) or with a nonradioactive molecule (biotin, acetylaminofluorene, digoxigenin, 5-bromodeoxyuridine, fluorescein) to obtain probes which are utilizable for numerous applications. Examples of nonradioactive labeling of nucleotide sequences are described, for example, in French Patent No. 78.10975 or by Urdea et al. or by Sanchez-Pescador et al. in 1988. In the latter case, it will also be possible to use one of the labeling methods described in patents FR-2 422 956 and FR-2 518 755.

The hybridization technique can be carried out in various manners (Matthews et al., 1988). The most general method consists in immobilizing the nucleic acid extract of cells on a support (such as nitrocellulose, nylon, polystyrene) and then incubating, under well-defined conditions, the immobilized target nucleic acid with the probe. After hybridization, the excess of probe is eliminated and the hybrid molecules formed are detected by the appropriate method (measurement of the radioactivity, of the fluorescence or of the enzymatic activity linked to the probe).

The disclosure likewise comprises the nucleotide sequences according to the disclosure, characterized in that they are immobilized on a support, covalently or noncovalently.

According to another advantageous mode of employing nucleotide sequences according to the disclosure, the latter can be used immobilized on a support and can thus serve to capture, by specific hybridization, the target nucleic acid obtained from the biological sample to be tested. If necessary, the solid support is separated from the sample and the hybridization complex formed between said capture probe and the target nucleic acid is then detected with the aid of a second probe, a so-called detection probe, labeled with an easily detectable element.

Another subject of the present disclosure is a vector for the cloning and/or expression of a sequence, characterized in that it contains a nucleotide sequence according to the disclosure.

The polypeptides according to the disclosure can likewise be prepared by techniques which are conventional in the field of the synthesis of peptides. This synthesis can be carried out in homogeneous solution or in solid phase. For example, reference can be made to the technique of synthesis in homogeneous solution described by Houben-Weyl in 1974. This method of synthesis consists in successively condensing, two by two, the successive amino acids in the order required, or in condensing amino acids and fragments formed previously and already containing several amino acids in the appropriate order, or alternatively several fragments previously prepared in this way, it being understood that it will be necessary to protect beforehand all the reactive functions carried by these amino acids or fragments, with the exception of amine functions of one and carboxyls of the other or vice-versa, which must normally be involved in the formation of peptide bonds, especially after activation of the carboxyl function, according to the methods well known in the synthesis of peptides. According to another preferred technique of the disclosure, recourse will be made to the technique described by Merrifield. To make a peptide chain according to the Merrifield procedure, recourse is made to a very porous polymeric resin, on which is immobilized the first C-terminal amino acid of the chain. This amino acid is immobilized on a resin through its carboxyl group and its amine function is protected. The amino acids which are going to form the peptide chain are thus immobilized, one after the other, on the amino group, which is deprotected beforehand each time, of the portion of the peptide chain already formed, and which is attached to the resin. When the whole of the desired peptide chain has been formed, the protective groups of the different amino acids forming the peptide chain are eliminated and the peptide is detached from the resin with the aid of an acid.

The disclosure additionally relates to hybrid polypeptides having at least one polypeptide according to the disclosure, and a sequence of a polypeptide capable of inducing an immune response in man or animals.

Advantageously, the antigenic determinant is such that it is capable of inducing a humoral and/or cellular response. It will be possible for such a determinant to comprise a polypeptide according to the disclosure in glycosylated form used with a view to obtaining immunogenic compositions capable of inducing the synthesis of antibodies directed against multiple epitopes. Said polypeptides or their glycosylated fragments are likewise part of the disclosure. These hybrid molecules can be formed, in part, of a polypeptide carrier molecule or of fragments thereof according to the disclosure, associated with a possibly immunogenic part, in particular an epitope of the diphtheria toxin, the tetanus toxin, a surface antigen of the hepatitis B virus (patent FR 79 21811), the VP1 antigen of the poliomyelitis virus or any other viral or bacterial toxin or antigen. The procedures for synthesis of hybrid molecules encompass the methods used in genetic engineering for constructing hybrid nucleotide sequences coding for the polypeptide sequences sought. It will be possible, for example, to refer advantageously to the technique for obtainment of genes coding for fusion proteins described by Minton in 1984. Said hybrid nucleotide sequences coding for a hybrid polypeptide as well as the hybrid polypeptides according to the disclosure characterized in that they are recombinant polypeptides obtained by the expression of said hybrid nucleotide sequences are likewise part of the disclosure.

The disclosure likewise comprises the vectors characterized in that they contain one of said hybrid nucleotide sequences. The host cells transformed by said vectors, the transgenic animals comprising one of said transformed cells as well as the procedures for preparation of recombinant polypeptides using said vectors, said transformed cells and/or said transgenic animals are, of course, likewise part of the disclosure.

The polypeptides according to the disclosure, the antibodies according to the disclosure described below and the nucleotide sequences according to the disclosure can advantageously be employed in procedures for the detection and/or identification of PEDV and/or ETEC in a biological sample (biological tissue or fluid) capable of containing them. These procedures, according to the specificity of the polypeptides, the antibodies and the nucleotide sequences according to the disclosure which will be used, will in particular be able to detect and/or to identify a PEDV and/or ETEC.

The polypeptides according to the disclosure can advantageously be employed in a procedure for the detection and/or the identification of PEDV and/or ETEC in a biological sample (biological tissue or fluid) capable of containing them, characterized in that it comprises the following steps: a) contacting of this biological sample with a polypeptide or one of its fragments according to the disclosure (under conditions allowing an immunological reaction between said polypeptide and the antibodies possibly present in the biological sample); and b) demonstration of the antigen-antibody complexes possibly formed. Preferably, the biological sample is formed by a fluid, for example a pig serum, whole blood or biopsies. Any conventional procedure can be employed for carrying out such a detection of the antigen-antibody complexes possibly formed. By way of example, a preferred method brings into play immunoenzymatic processes according to the ELISA technique, by immunofluorescence, or radioimmunological processes (RIA) or their equivalent.

Thus, the disclosure likewise relates to the polypeptides according to the disclosure, labeled with the aid of an adequate label such as of the enzymatic, fluorescent or radioactive type. Such methods comprise, for example, the following steps: 1) deposition of determined quantities of a polypeptide composition according to the disclosure in the wells of a microtiter plate; 2) introduction into said wells of increasing dilutions of serum, or of a biological sample other than that defined previously, having to be analyzed; 3) incubation of the microplate; and 4) introduction into the wells of the microtiter plate of labeled antibodies directed against pig immunoglobulins, the labeling of these antibodies having been carried out with the aid of an enzyme selected from those which are capable of hydrolyzing a substrate by modifying the absorption of the radiation of the latter, at least at a determined wavelength, for example at 550 nm, detection, by comparison with a control test, of the quantity of hydrolyzed substrate.

The disclosure likewise relates to a kit or set for the detection and/or identification of PEDV and/or ETEC, characterized in that it comprises the following elements: 1) a polypeptide according to the disclosure; 2) if need be, the reagents for the formation of the medium favorable to the immunological or specific reaction; 3) if need be, the reagents allowing the detection of the antigen-antibody complexes produced by the immunological reaction between the polypeptide(s) of the disclosure and the antibodies possibly present in the biological sample, these reagents likewise being able to carry a label, or to be recognized in their turn by a labeled reagent, more particularly in the case where the polypeptide according to the disclosure is not labeled; 4) if need be, a biological reference sample (negative control) devoid of antibodies recognized by a polypeptide according to the disclosure; and 5) if need be, a biological reference sample (positive control) containing a predetermined quantity of antibodies recognized by a polypeptide according to the disclosure.

The polypeptides according to the disclosure allow monoclonal or polyclonal antibodies to be prepared which are characterized in that they specifically recognize the polypeptides according to the disclosure. It will advantageously be possible to prepare the monoclonal antibodies from hybridomas according to the technique described by Kohler and Milstein in 1975. It will be possible to prepare the polyclonal antibodies, for example, by immunization of an animal, in particular a mouse, with a polypeptide or a DNA, according to the disclosure, associated with an adjuvant of the immune response, and then purification of the specific antibodies contained in the serum of the immunized animals on an affinity column on which the polypeptide which has served as an antigen has previously been immobilized. The polyclonal antibodies according to the disclosure can also be prepared by purification, on an affinity column on which a polypeptide according to the disclosure has previously been immobilized, of the antibodies contained in the serum of pigs infected by a PEDV and/or ETEC.

The disclosure likewise relates to mono- or polyclonal antibodies or their fragments, or chimeric antibodies, characterized in that they are capable of specifically recognizing a polypeptide according to the disclosure. It will likewise be possible for the antibodies of the disclosure to be labeled in the same manner as described previously for the nucleic probes of the disclosure, such as a labeling of enzymatic, fluorescent or radioactive type.

The disclosure is additionally directed at a procedure for the detection and/or identification of PEDV and/or ETEC, in a biological sample, characterized in that it comprises the following steps: a) contacting of the biological sample (biological tissue or fluid) with a mono- or polyclonal antibody according to the disclosure (under conditions allowing an immunological reaction between said antibodies and the polypeptides of PEDV and/or ETEC, possibly present in the biological sample); and b) demonstration of the antigen-antibody complex possibly formed.

Likewise within the scope of the disclosure is a kit or set for the detection and/or the identification of PEDV and/or ETEC, characterized in that it comprises the following components: a) a polyclonal or monoclonal antibody according to the disclosure, if need be labeled; b) if need be, a reagent for the formation of the medium favorable to the carrying out of the immunological reaction; c) if need be, a reagent allowing the detection of the antigen-antibody complexes produced by the immunological reaction, this reagent likewise being able to carry a label, or being capable of being recognized in its turn by a labeled reagent, more particularly in the case where said monoclonal or polyclonal antibody is not labeled; and d) if need be, reagents for carrying out the lysis of cells of the sample tested.

The present disclosure likewise relates to a procedure for the detection and/or the identification of PEDV and/or ETEC in a biological sample, characterized in that it employs a nucleotide sequence according to the disclosure. More particularly, the disclosure relates to a procedure for the detection and/or the identification of PEDV and/or ETEC, in a biological sample, characterized in that it contains the following steps: a) if need be, isolation of the DNA from the biological sample to be analyzed; b) specific amplification of the DNA of the sample with the aid of at least one primer, or a pair of primers, according to the disclosure; and c) demonstration of the amplification products. These can be detected, for example, by the technique of molecular hybridization utilizing a nucleic probe according to the disclosure. This probe will advantageously be labeled with a nonradioactive (cold probe) or radioactive element.

For the purposes of the present disclosure, “DNA of the biological sample” or “DNA contained in the biological sample” will be understood as meaning either the DNA present in the biological sample considered, or possibly the cDNA obtained after the action of an enzyme of reverse transcriptase type on the RNA present in said biological sample.

Reduction in the incidence of and/or severity refers to a therapeutic or protective immunological response such that resistance to new infection will be enhanced and/or the clinical severity of the disease reduced. Such protection will be demonstrated by either a reduction in the severity or prevalence of, up to and including a complete lack of symptoms normally displayed by an infected host, a quicker recovery time and/or a lowered viral titer in the infected host. The reduction will be in comparison to an animal or a group of animals that did not receive an administration of a composition disclosed herein.

According to a further aspect, the present disclosure provides a multivalent combination vaccine which includes an immunological agent effective for reducing the incidence of or lessening the severity of PEDV and/or ETEC infection, and at least one immunological active component against another disease-causing organism in swine.

Preferably the other disease-causing organism in swine is selected from the group consisting of: Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhsiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa; Leptospira icterohaemorrhagicae; and Leptospira interrogans; Leptospira pomona; Leptospira tarassovi; Mycobacterium spp. preferably M. avium; M. intracellulare; and M. bovis; Mycoplasma hyopneumoniae (M hyo); Pasteurella multocida; Porcine cytomegalovirus; Porcine Parvovirus; Porcine Reproductive and Respiratory Syndrome (PRRS) Virus; Pseudorabies virus; Rotavirus; Salmonella spp.; preferably S. thyhimurium; and S. choleraesuis; Staph. hyicus; Staphylococcus spp. preferably Streptococcus spp., preferably Strep. suis; Swine herpes virus; Swine Influenza Virus; Swine pox virus; Swine pox virus; Vesicular stomatitis virus; Virus of vesicular exanthema of swine; Leptospira Hardjo; and/or Mycoplasma hyosynoviae.

The disclosure is likewise directed at a pharmaceutical composition according to the disclosure, for the prevention or the treatment of an infection by a PEDV and/or ETEC.

It is understood that “prevention” as used in the present disclosure, includes the complete prevention of infection by a PEDV and/or ETEC, but also encompasses a reduction in the severity of or incidence of clinical signs associated with or caused by PEDV and/or ETEC infection. Such prevention is also referred to herein as a protective effect.

The disclosure likewise concerns the use of a composition according to the disclosure, for the preparation of a medicament intended for the prevention or the treatment of infection by a PEDV and/or ETEC.

These compounds can be administered by the systemic route, in particular by the intravenous route, by the intramuscular, intradermal or subcutaneous route, or by the oral route. In a more preferred manner, the vaccine composition comprising polypeptides according to the disclosure will be administered by the intramuscular route, through the food or by nebulization several times, staggered over time.

Their administration modes, dosages and optimum pharmaceutical forms can be determined according to the criteria generally taken into account in the establishment of a treatment adapted to an animal such as, for example, the age or the weight, the seriousness of its general condition, the tolerance to the treatment and the secondary effects noted. Preferably, the vaccine of the present disclosure is administered in an amount that is protective or provides a protective effect against PEDV and/or ETEC infection.

For example, in the case of a vaccine according to the present disclosure comprising a polypeptide encoded by a nucleotide sequence of the genome of PEDV and/or ETEC, or a homolgue or fragment thereof, the polypeptide will be administered one time or several times, spread out over time, directly or by means of a transformed cell capable of expressing the polypeptide, in an amount of about 0.1 to 10 μg per kilogram weight of the animal, preferably about 0.2 to about 5 μg/kg, more preferably about 0.5 to about 2 μg/kg for a dose.

An immunologically effective amount of the vaccines or immunogenic compositions of the present disclosure is administered to a pig in need of protection against infection by PEDV and/or ETEC. Preferably, the immunogenic composition will decrease the severity or incidence of at least one clinical sign or symptom associated with PEDV and/or ETEC infection. In some forms, the clinical sign or symptom is selected from the group consisting of diarrhea, vomiting, anorexia death, dehydration, and any combination thereof for PEDV and diarrhea, vomiting, dehydration, roughened hair coat, subnormal body temperature, shivering, death, neonatal septicemia and polyserositis, lesions, excess watery fluid in the small intestine and/or colon, a distended and/or gas-filled small intestine and/or colon, mild reddening and congestion of the stomach, congestion of the gastrointestinal tract, coliforms adhered to microvilli of intestinal epithelial cells, necrosis of villi, microvascular thrombosis in the lamina propria, fibrinous polyserositis, arthritis, and any combination thereof for ETEC. The immunologically effective amount or the immunogenic amount that inoculates the pig can be easily determined or readily titrated by routine antigen-specific antibody titer testing. An effective amount is one in which a sufficient immunological response to the vaccine is attained to protect the pig exposed to PEDV and/or ETEC. Preferably, the pig is protected to an extent in which one to all of the adverse physiological symptoms or effects of the viral disease are significantly reduced, ameliorated or totally prevented.

The vaccine can be administered in a single dose or in repeated doses with single doses being preferred. Single dose vaccines provide protection after a single dose without the need for any booster or subsequent dosages. Protection can include the complete prevention of clinical signs of infection, or a lessening of the severity, duration, or likelihood of the manifestation of one or more clinical signs of infection. Methods are known in the art for determining or titrating suitable dosages of active antigenic agent to find minimal effective dosages based on the weight of the pig, concentration of the antigen and other typical factors.

Desirably, the vaccine is administered to a pig not yet exposed to the PEDV and/or ETEC. The vaccine containing the antigenic forms thereof can conveniently be administered intranasally, transdermally (i.e., applied on or at the skin surface for systemic absorption), parenterally, etc. The parenteral route of administration includes, but is not limited to, intramuscular, intravenous, intraperitoneal, intradermal (i.e., injected or otherwise placed under the skin) routes and the like. Although less convenient, it is also contemplated that the vaccine is given to the pig through the intralymphoid route of inoculation.

When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

In one or more embodiments, the invention is concerned with an immunogenic agent comprising (consisting essentially of or consisting of) a chimeric antigen-adjuvant protein. The chimeric antigen-adjuvant protein comprises (consists essentially of or consists of) an enterotoxin sequence having one or more segments substituted (i.e., replaced) with amino acid residues (fragments) of one or more epitopes. Preferably, the enterotoxin is a detoxified, bacterial heat-labile enterotoxin or subunit thereof, and more preferably the enterotoxin is E. coli heat-labile LT toxin or subunit thereof. Preferred enterotoxins will induce a strong mucosal immune response, such as by binding to the ganglioside GMI receptor on host epithelial cells. Accordingly, it will be appreciated that the inventive immunogenic agents are advantageously self-adjuvants, and do not require the administration of a separate adjuvant or adjuvant system. In one or more embodiments, the compositions, systems, and methods described herein do not use and are free of separate adjuvants. However, in other embodiments, the compositions, systems, and methods disclosed herein incorporate or use an additional adjuvant. The term “adjuvant” is used herein to refer to substances that have immunopotentiating effects and are added to or co-formulated with an active agent in order to enhance the immune response against the active agent.

The segment(s) to be replaced in the enterotoxin sequence will be selected such that the substituted epitope fragments will be presented on the outside of the enterotoxin surface, once the protein undergoes (re)folding. Therefore, it will be appreciated that unlike previous fusion proteins, the chimeric antigens are not simply end-to-end fusions of two different proteins, or insertions of one protein sequence into another. That is, the position of the substitution will be selected based upon the physical folded structure of the enterotoxin, which can be predicted using various publicly-available databases and software. The enterotoxin segment will then be replaced accordingly with an epitope fragment to be presented on the surface of the folded enterotoxin protein. In one or more embodiments, the epitope fragments consist of from about 7 to about 20 amino acids. In one or more embodiments, the enterotoxin is substituted by multiple different epitope fragments, for example from about 1 epitope fragment up to about 12 different epitope fragments, to create multi-valent chimeric proteins (aka multi-epitope fusion antigen MEFA). In one or more embodiments, the epitope fragments are synthesized into a multi-epitope construct, which is then substituted into the enterotoxin. The multi-epitope construct can include linker peptide sequences and spacers to facilitate flexibility and presentation of the epitopes. In one or more embodiments, the enterotoxin is substituted with at least one B-cell epitope and at least one T-cell epitope. In one or more embodiments, the epitopes fragments are derived from immunogenic targets that elicit an immune response against the target disease.

In one or more embodiments, the target disease is PEDV and the epitope fragments are selected from the group consisting of the epitopes: HGKVVSNQPLLVN (SEQ ID NO.1), GPRLQPYEVFEKV (SEQ ID NO.2), YSNIGVCK (SEQ ID NO.3), SQSGQVKI (SEQ ID NO.4), PYYCFFKVDTYNSTVYKFL (SEQ ID NO.5), and combinations thereof as well as sequences having at least 80, 85, 90, 91, 92, 93, 94, 95, 96, 97, 98, 99, or 99.5% sequence homology with such sequences.

In one or more embodiments, the target disease is ETEC and the epitope fragments are selected from the group consisting of continuous B cell epitope subunits of LTA, K88 FaeG, F18 adhesin, and any combination thereof. In some forms, the ETEC subunits are selected from the group consisting any one or more of SEQ ID NOS. 9-35 as well as sequences having at least 80, 85, 90, 91, 92, 93, 94, 95, 96, 97, 98, 99, or 99.5% sequence homology with such sequences. In some forms, the continuous B cell epitope subunits are selected from the K88 and F18 subunits thereof, as well as any combination thereof. In some forms, the continuous B cell epitope subunits are selected from the group consisting of SEQ ID NOS. 23, 24, 31, 35, and any combination thereof.

The nucleic acid sequence of the chimeric protein is preferably a nucleic acid sequence that can be expressed in large amounts in an E. coli host-vector system, and the expressed proteins retain the functional characteristics of an epitope and an adjuvant.

Advantageously, the immunogenic agent can be transformed into a live, non-pathogenic E. coli strain (preferably using an appropriate plasmid), and administered to a subject to express the chimeric antigen-adjuvant protein in a subject. In one or more embodiments, the E. coli strain is species-specific for the subject, for colonization of the subject and continuous expression of the immunogenic agent. In one or more embodiments, the E. coli strain is selected so that it will recognize fimbrial receptors at epithelial cells and establish colonization.

In one or more embodiments, the invention is also concerned with a vaccine composition for treating and/or preventing PEDV, comprising an effective amount of the immunogenic agent and a pharmaceutically acceptable carrier. In one or more embodiments, the invention is also concerned with methods of treating and/or preventing PEDV, comprising administration of an immunogenic agent to a pig. The immunogenic agent can be administered using any suitable route, including oral administration. Oral dosage forms include, without limitation, capsules, tablets, emulsions, water suspensions, dispersants, and solutions. One of skill in the art recognizes that an amount may be considered therapeutically “effective” even if the condition is not totally eradicated or prevented, but it or its symptoms and/or effects are improved or alleviated partially in the subject. In some embodiments, the agent or compositions can be provided in unit dosage form in a suitable container. The term “unit dosage form” refers to a physically discrete unit suitable as a unitary dosage for human or animal use. Each unit dosage form may contain a predetermined amount of the immunogenic agent (and/or other active agents) in the carrier calculated to produce a desired effect.

The terms “therapeutic” or “treat,” as used herein, refer to processes that are intended to produce a beneficial change in an existing condition (e.g., viral infection, disease, disorder) of a subject, such as by reducing the severity of the clinical symptoms and/or effects of the infection, and/or reducing the duration of the infection/symptoms/effects. The terms “prophylactic” or “prevent,” as used herein, refer to processes that are intended to inhibit or ameliorate the effects of a future viral infection or disease to which a subject may be exposed (but is not currently infected with). In some cases the composition may prevent the development of observable morbidity from viral infection (i.e., near 100% prevention). In other cases, the composition may only partially prevent and/or lessen the extent of morbidity due to the viral infection (i.e., reduce the severity of the symptoms and/or effects of the infection, and/or reduce the duration of the infection/symptoms/effects). In either case, the agents are still considered to “prevent” the target infection or disease.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

The following examples demonstrate certain aspects of the present disclosure. However, it is to be understood that these examples are for illustration only and do not purport to be wholly definitive as to conditions and scope of this disclosure. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified. Further unless noted otherwise, all components of the disclosure are understood to be disclosed to cover “comprising”, “consisting essentially of”, and “consisting of” claim language as those terms are commonly used in patent claims.

### EXAMPLE 1

### Porcine Epidemic Diarrhea Virus

We identified five epitopes from the PEDV spike protein, and embedded these PEDV epitopes into a monomeric heat-labile (LT) toxoid or a holotoxin-structured LT for LT-PEDV MEFA (multi-epitope fusion antigen). The epitope-toxin chimera were expressed in E. coli as a recombinant protein or a live strain expressing the holotoxin-structured antigen. LT-PEDV MEFA has been expressed, and the monomeric protein induced antibodies to PEDV S protein. The holotoxin-structured LT-PEDV MEFA has also been expressed in a live nonpathogenic E. coli field strain. Mice orally inoculated with this live E. coli strain developed neutralizing antibodies against PEDV, as shown in the data below.

1) PEDV epitopes in silico prediction. B-cell epitopes of PEDV S protein were predicted in silico (using computer-based software or adapted from previous studies). Predicted B-cell epitopes were subjects to be fused to the strongly immunogenic monomeric Escherichia coli heat-labile toxoid monomer (one LTA subunit and one LTB subunit forming a single peptide) or the native eltAB genes coding the holotoxin-structured LT (one LTA and five LTB forming a holotoxin).

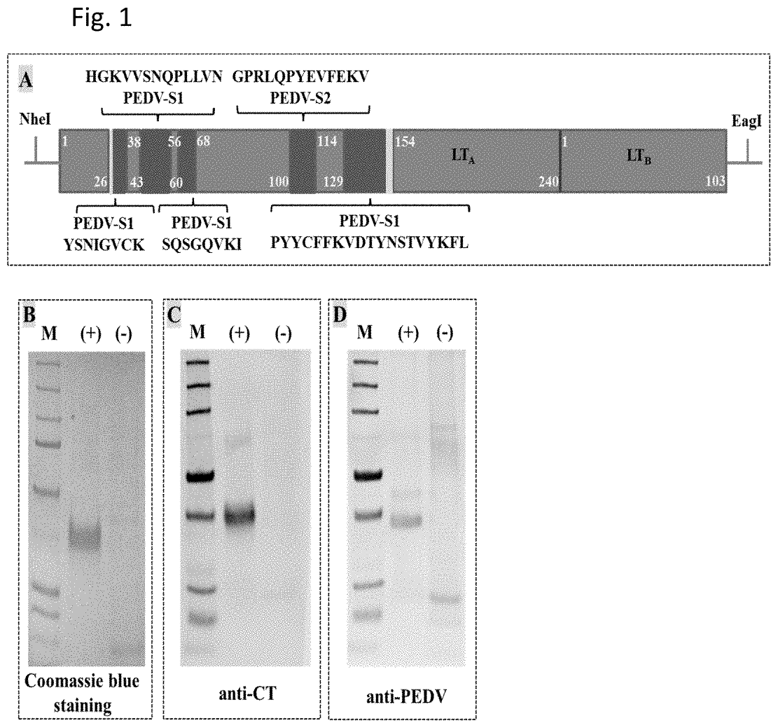

2) Monomeric PEDV-LTR192G MEFA construction & detection. Predicted PEDV S protein epitopes were first embedded into the A1 peptide of a non-toxic E. coli heat-labile toxoid LTR192G monomer (FIG. 1A). The fragment coding the first 160 amino acids of the LT-PEDV chimeric fusion genes, which contains the PEDV epitopes, was synthesized by IDT (Integrated DNA Technology, IA). This synthetic fragment was overlapped with the LTR192G monomer using SOE (splice-overlap-extension) PCRs. Resultant PEDV-LT fusion gene was cloned into pET28α and expressed in E. coli BL21 strain.

Expression of the LT-PEDV fusion protein was examined in sodium dodecyl sulfate polyacrylamide gel electrophoresis (SDS-PAGE) with Coomassie blue staining (FIG. 1B), and immune blot assays using anti-LT and anti-PEDV antiserum (FIG. 1C).

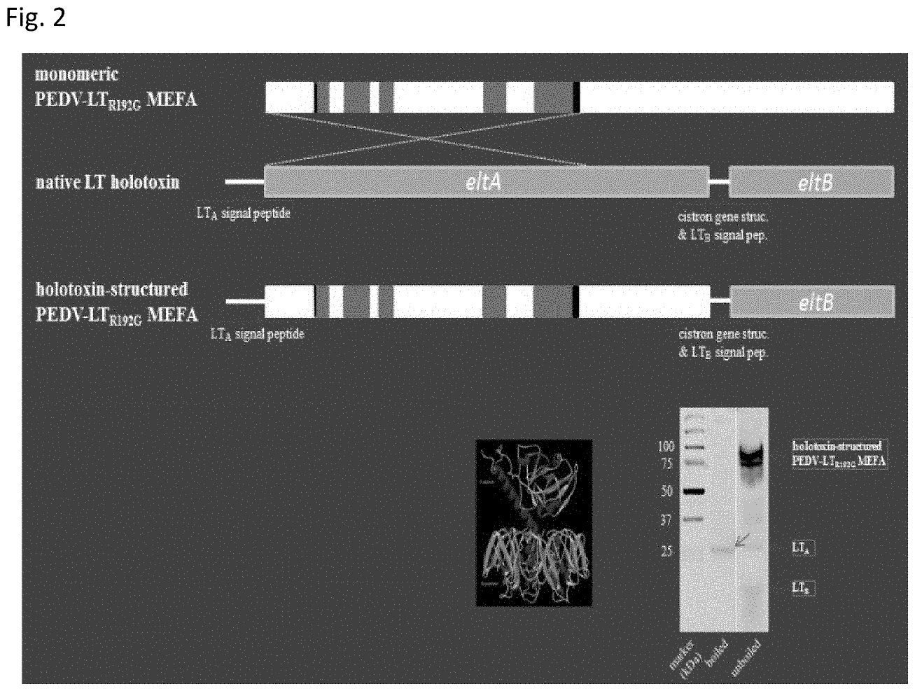

3) Holotoxin-structured PEDV-LTR192G MEFA construction: The 5′ end fragment coding the first 192 amino acids of eltAB gene coding the holotoxin LT was substituted with the fragment coding the first 192 amino acids of the monomeric PEDV-LTR192G using SOE PCRs (FIG. 2). Resultant LT-like PEDV-LTR192G fusion genes were cloned into vector pBR322 to generate pPEDV-LTR192G plasmid. Plasmid pPEDV-LTR192G was introduced into nonpathogenic porcine E. coli field isolate 1836-2.

4) Construction of a safe and effective live PEDV vaccine strain. Expression of LT-like holotoxin-structured PEDV-LTR192G protein by 1836-2 was examined in SDS-PAGE immune blot using anti-CT antibodies (Sigma). A protein at a size about 85 kDa, equivalent to the holotoxin protein was detected by anti-CT antiserum (FIG. 2). Expression of this holotoxin-structured fusion protein was verified in GM ELISA. This strain was designated as the live PEDV-LTR192G vaccine strain.

5) Mouse immunization: Adult female BALB/C mice (Charles River Laboratories International, Inc., Wilmington, Mass.), 6 per group, were intraperitoneally immunized with 150 μg recombinant PEDV-LTR192G monomeric protein, in an equal volume of Freund's incomplete adjuvant (Sigma). Two boosters at the same dose of the primary were followed at an interval of two weeks. A group of 6 mice without immunization was served as the control. Ten days after the second booster, mice in the immunized group and the control group were anaesthetized with CO2 and then exsanguinated. Serum samples were collected and stored at −800□C until use.

6) Antibody titration: Serum samples collected from each immunized and control mouse were used to titrate anti-LT and anti-PEDV specific antibodies in ELISAs. Cholera toxin (CT; Sigma, St. Louis, Mo.), recombinant S1 and S2 proteins, were used to coat wells of 2HB microtiter plates (100 ng/well) at 37° C. for 1 h and then 4° C. overnight. Coated wells were washed with PBST, and uncoated spots were blocked with 5% non-fat milk. Plates were washed, and incubated with serially two-fold diluted serum samples from each immunized or control mouse, at 37° C. for 1 h. After washes, wells were incubated with 1:3300 horse-radish peroxidase (HRP) conjugated goat-anti-mouse IgG (Sigma) at 37° C. for 1 h. Wells were washed and incubated with 3,3′,5,5′-tetramethylbenzidine (TMB) Microwell Peroxidase Substrate (KPL, Gaithersburg, Md.) at room temperature for 20 min. Optical densities (OD) were measured with a plate reader at the wavelength of 405 nm. Antibody titers were calculated from the highest serum dilutions that gave OD readings above 0.3 (after subtraction of the background readings) and were presented in log10.

7) Antibody neutralization: Induced anti-PEDV antibodies were examined for neutralization activity in serum samples using a modified plaque reduction neutralization assay. Serum samples pooled from each group of immunized mice, in 2-fold serial dilution, were mixed with PEDV 1×105 TCID50/ml and incubated. The mixture (serum sample with virus) was added to confluent monolayers of Vero cells in a 96-well plate. Cells were further incubated in Eagle minimal medium and visualized microscopically.

I. LT-PEDV MEFA DNA sequences, protein expression & detection:

```
TTGACATCATGTTGCATATAGGTTAGATAAAACAAGTGGCGTTATCTTTTTCCGGATTGTCTTCTTGTATGATATATAAGT
TTTCCTCGATGAAAAATATAACTTTCATTTTTTTTATTTTATTAGCATCGCCATTATATGCAAATGGCGACAGATTATACCG
TGCTGACTCTAGACCCCCAGATGAAATAAAACGTTCCGGAGGTCTTATGCC
                                                LT (with signal sequence) 1-218
CAGAGGAGGTGGAGGAAGCTATAGTAACATAGGTGTTTGTAAAATTAATCTTTATGATCATGGTAAGGT
  Linker: GGGGS (SEQ ID NO. 6) PEDV S 751-758: YSNIGVCK (SEQ ID NO. 3) LT 258-272
GGTTTCCAACCAACCATTGTTGGTCAATGACGGATATGTTTCTCAGTCTGGCCAAGTCAAGATTGCTCACT
PEDV S 270-282: HGKVVSNQPLLVN (SEQ ID NO. 1) LT 312-323 PEDV S 767-774: SQSGQVKI (SEQ ID
NO. 4)
TAGCAGGACAGTCTATATTATCAGGATATTCCACTTACTATATATATGTTATAGCGACAGCACCAAATATG
                                              LT 348-455
TTTAATGTTAATGATGTATTAGGCGTATACGGTCCTAGACTTCAACCTTACGAAGTTTTTGAAAAGGTCGG
                                                            PEDV S 1371-1383: GPRLQPYEVFEKV (SEQ ID NO. 2)
AATACCATATTCTCAGATATATGGATGGTATCGTGTTAATTTTGGTCCCTATTATTGTTTTTTTAAAGTGGA
LT 495-542 PEDV S 370-388: PYYCFFKVDTYNSTVYKFL (SEQ ID NO. 5
TACTTACAACTCCACTGTTTATAAATTCTTAGGACCCGGACCTGGTGCAGAAGATGGTTACAGATTAGCA
                                            Linker: GPGPG (SEQ ID NO. 7) LT 615---end
GGTTTCCCACCGGATCACCAAGCTTGGAGAGAAGAACCCTGGATTCATCATGCAC
CACAAGGTTGTGGAAATTCATCAAGAACAATCACAGGTGATACTTGTAATGAGGA
GACCCAGAATCTGAGCACAATATATCTCAGGGAATATCAATCAAAAGTTAAGAG
GCAGATATTTTCAGACTATCAGTCAGAGGTTGACATATATAACAGAATTCGGGAT
GAATTATGAATAAAGTAAAATGTTATGTTTTATTTACGGCGTTACTATCCTCT
CTATATGCACACGGAGCTCCCCAGACTATTACAGAACTATGTTCGGAATATCGC
AACACACAAATATATACGATAAATGACAAGATACTATCATATACGGAATCGATG
GCAGGCAAAAGAGAAATGGTTATCATTACATTTAAGAGCGGCGAAACATTTCAG
GTCGAAGTCCCGGGCAGTCAACATATAGACTCCCAGAAAAAAGCCATTGAAAGG
ATGAAGGACACATTAAGAATCACATATCTGACCGAGACCAAAATTGATAAATTAT
GTGTATGGAATAATAAAACCCCCAATTCAATTGCGGCAATCAGTATGAAAAACTA
G (SEQ ID NO. 8)
```

### EXAMPLE 2

This Example prepares and tests a broadly effective vaccine against porcine post-weaning diarrhea

In silico antigenic epitope prediction

Because ETEC toxin STa, STb and Stx2e epitopes have already been identified and confirmed for induction of neutralizing antibodies against STa enterotoxicity or STb and Stx2e cytotoxicity, as well as for protection against diarrhea from challenge of an ETEC strain producing STa, STb and LT toxins (Rausch et al. 2017, Vet. Microbiol. 202(2017):79-89), this example focused on epitope in silico identification for ETEC heat-labile toxin (LT) and ETEC fimbriae K88 and F18.

A total of eleven continuous B-cell epitopes were identified in silico from LT_{A} subunit which was intended to carry and to present heterogeneous epitopes, ranging from 10 to 13 amino acid residues (Table 1).

TABLE 1
LTA subunit continuous B-cell epitopes were identified from epitope
prediction programs (Larsen, 2006 #41490)(Saha, 2007 #10867), with
epitope amino acid sequence, position, and length indicated.

| epitope | aa sequence | Position | Length | |
|---|---|---|---|---|
| e1 | TITGDTCNEETQ | 193-204 | 12 | SEQ ID NO. 9 |
| e2 | NGDKLYRADSR | 1-11 | 11 | SEQ ID NO. 10 |
| e3 | DSRPPDEIKRSGG | 9-21 | 13 | SEQ ID NO. 11 |
| e4 | RGHNEYFDRGTQ | 25-36 | 12 | SEQ ID NO. 12 |
| e5 | YDHARGTQTG | 42-51 | 10 | SEQ ID NO. 13 |
| e6 | RYDDGYVSTS | 54-63 | 10 | SEQ ID NO. 14 |
| e7 | SPIWYEQEVSA | 105-115 | 11 | SEQ ID NO. 15 |
| e8 | ITRNREYRDRY | 140-149 | 10 | SEQ ID NO. 16 |
| e9 | APAEDGYRLAG | 156-166 | 11 | SEQ ID NO. 17 |
| e10 | AGFPPDHQAWREE | 165-177 | 13 | SEQ ID NO. 18 |
| e11 | ITHAPQGCGNSSRT | 181-193 | 13 | SEQ ID NO. 19 |

Ten epitopes (e2 to e11) located at the enzymatic LT A1 domain and one epitope (e1) was from the LT A2 domain. Computation modeling using Phyre2 showed all epitopes were surface exposed (FIGS. 1A & 1D). This LT_{A} protein model was deposited as PDBID:1LTA. All predicted epitopes were located at α-helix or β-sheet of protein secondary structure (FIGS. 1B & 1C).

K88 major structural subunit FaeG is also the adhesin which attaches to host receptors. Neutralizing antibodies against FaeG are expected to be protective against K88 fimbrial adherence to pig receptors. A total of 9 epitopes, ranged from 10 aa to 16 residues were identified from the FaeG protein (FIG. 2)(Table 2).

TABLE 2
K88 fimbrial subunit FaeG continuous B-cell epitopes, with
epitope amino acid sequence, position, and length indicated.

| epitope | aa sequence | Position | Length | |
|---|---|---|---|---|
| K88e1 | MTGDFNGSVD | 31-40 | 10 | SEQ ID NO. 20 |
| K88e2 | LNDLTNGGTK | 69-78 | 10 | SEQ ID NO. 21 |
| K88e3 | GRTKEAFATP | 92-101 | 10 | SEQ ID NO. 22 |
| K88e4 | ELRKPDGGTN | 123-132 | 10 | SEQ ID NO. 23 |
| K88e5 | PMKNAGGTKVGSVKVN | 141-156 | 16 | SEQ ID NO. 24 |
| K88e6 | GRGGVTSADGEL | 164-175 | 12 | SEQ ID NO. 25 |
| K88e7 | PRGSELSAGSA | 192-202 | 11 | SEQ ID NO. 26 |
| K88e8 | RENMEYTDGT | 240-249 | 10 | SEQ ID NO. 27 |
| K88e9 | FNQAVTTSTQ | 269-278 | 10 | SEQ ID NO. 28 |

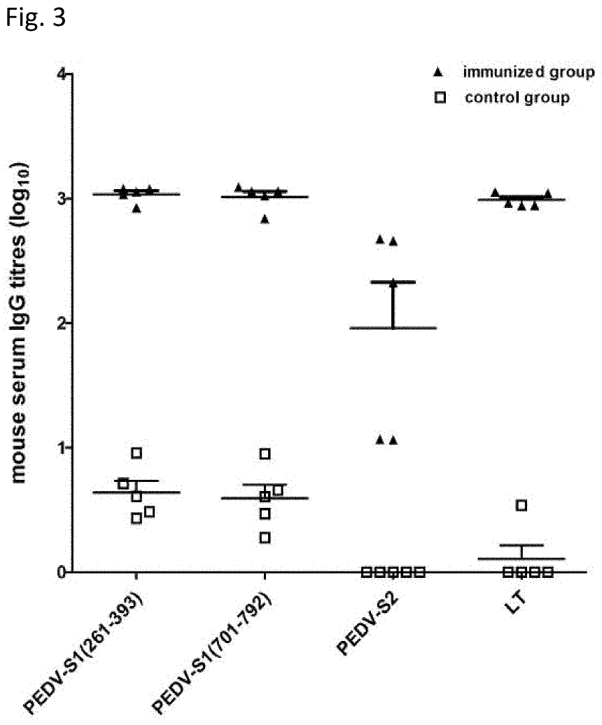

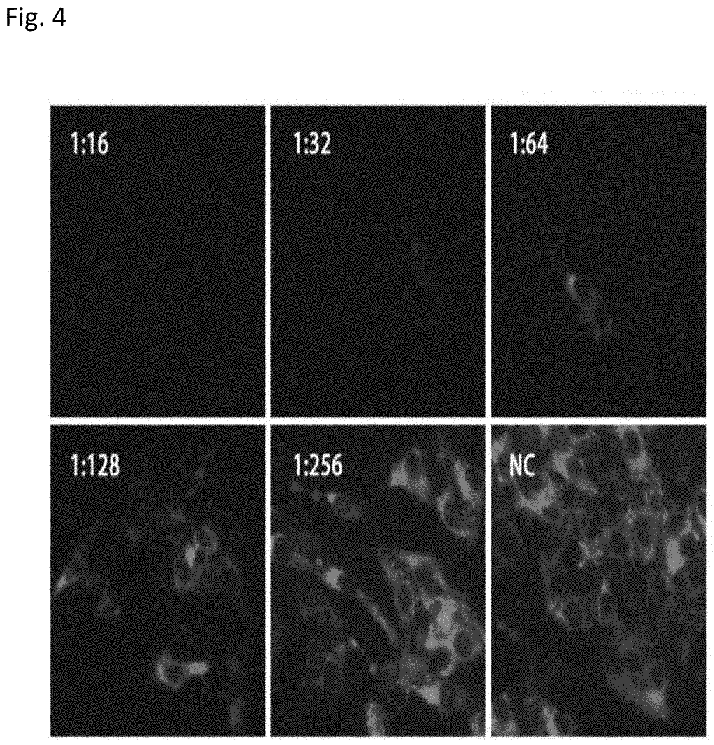

A total of seven continuous B-cell epitopes were identified from F18 adhesin subunit FedF (FIG. 3) (Table 3).

TABLE 3
F18 adhesin subunit FedF continuous B-cell epitopes, with epitope
amino acid sequence, position, and length indicated.

| epitope | aa sequence | Position | Length | |
|---|---|---|---|---|
| F18e1 | INSSASSAQV | 34-43 | 10 | SEQ ID NO. 29 |
| F18e2 | LGTGKTNTTQM | 48-58 | 11 | SEQ ID NO. 30 |
| F18e3 | IPSSSGTLTCQAGT | 74-87 | 14 | SEQ ID NO. 31 |
| F18e4 | NESQWGQQSQ | 115-124 | 10 | SEQ ID NO. 32 |
| F18e5 | AQTYPLSSGD | 151-160 | 10 | SEQ ID NO. 33 |
| F18e6 | PNQNDMPSSN | 226-235 | 10 | SEQ ID NO. 34 |
| F18e7 | QPDATGSWYD | 253-262 | 10 | SEQ ID NO. 35 |

Epitope-LT_{R192G} fusion and antigenicity

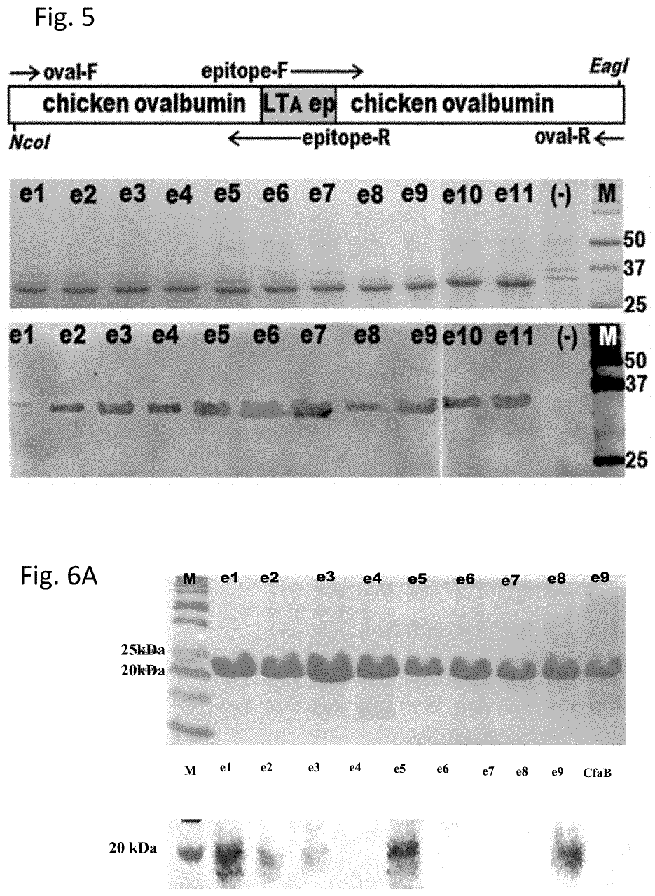

LT epitope and ovalbumin fusion construction, expression, and mouse immunization. To eliminate cross reactivity from carrier protein LT_{R192G} (which was designed originally to carry each LT_{A} epitope), the research plan was modified by selecting chicken ovalbumin protein (instead of LT_{R192G}) as the carrier to carry and to present each LT epitope. This ovalbumin carrier protein was verified for no reactivity with anti-LT or anti-CT antibodies. Each LT_{A} epitope was genetically fused to a modified chicken ovalbumin gene (with introns truncated) by replacing an ovalbumin epitope for epitope-ovalbumin fusions (FIG. 5). DNA sequencing verified that each LT_{A} epitope was correctly inserted into ovalbumin gene. Computational modeling indicated each LT_{A} epitope was surface-exposed and epitope replacement did not affect ovalbumin protein structure or protein secondary structure stability. Each epitope-ovalbumin fusion gene was cloned into pET28a vector and expressed in E. coli BL21. Recombinant proteins were 6× His-tag-less and shown an expected size of 34 kDa (FIG. 5).

Eleven epitope-ovalbumin fusion proteins were extracted from recombinant strains. Each fusion protein was recognized by anti-LT antiserum in Western blot, indicating these LT epitopes maintained their native antigenicity (FIG. 5). When immobilized on wells of ELISA plates, all epitope-ovalbumin fusion proteins reacted with anti-LT antiserum at a similar level, indicated by similar OD readings. After antigenicity verification in Western blot and GM1 ELISA, each fusion protein was used in mouse immunization to examine epitope immunogenicity. Eight-week-old female BALB/c mice (Charles River Laboratories International, Inc., Wilmington, Mass.), five per group, were subcutaneously immunized with each epitope-ovalbumin fusion protein (40 μg fusion protein in 25 μl PBS mixed with adjuvant −25 μl Montanide ISA 51 VG (SEPPIC, Fairfield, N.J.)). Five mice immunized with the modified chicken ovalbumin protein and ISA adjuvant and another group of five mice immunized with sterile PBS were used as two control groups. Immunized mice received two boosters at the same dose of the primary in an interval of two weeks. Mice were euthanized two weeks after the second booster. Mouse serum samples were collected for anti-LT antibody titration.

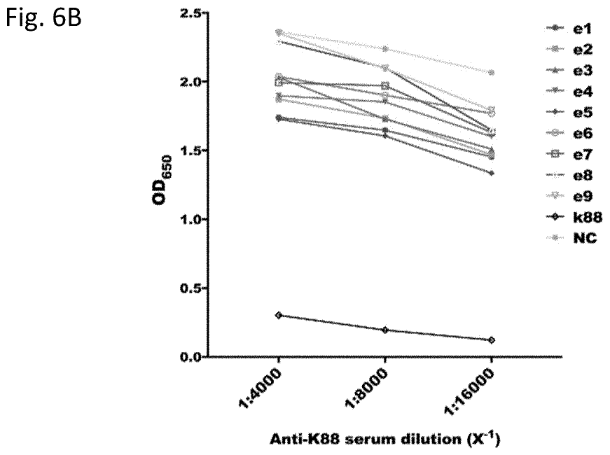

K88 FaeG epitope fusion protein expression, extraction and mouse immunization. Each FaeG epitope was genetically inserted to non-homologous human ETEC CFA/I adhesin subunit CfaB using splicing overlap extension (SOE) PCR. PCR amplified epitope fusion genes were digested with NheI and EagI, and were cloned into expression vector pET28a and expressed in E. coli BL21. Epitope fusion recombinant proteins were extracted (FIG. 6A). When examined in a standard sodium dodecyl sulfate-polyacrylamide gel electrophoresis (SDS-PAGE) Western blot using anti-K88 antiserum, only fusions with epitope 1, 2, 3, 5, or 9 were recognized (FIG. 6A). However, all fusion recombinant proteins were recognized in ELISA with anti-K88 antiserum (FIG. 6B).

With protein antigenicity verified, each FaeG epitope fusion protein was used in mouse immunization. Eight-week old BALB/c mouse (5 mice per group) were each subcutaneously immunized with 40 μg epitope-CfaB fusion protein and 0.2 μg double mutant LT (dmLT) adjuvant. Immunized mice received two booster injections (the same dose of the primary) biweekly. Two weeks after the second booster injection, mice were euthanized. A group of five mice without immunization was used as the control. Mouse serum samples were collected and stored at −80° C. until use.

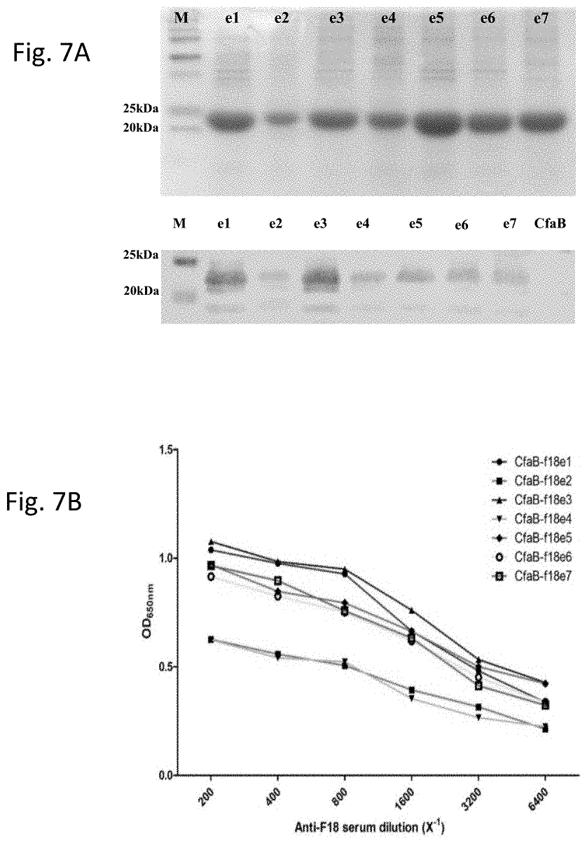

F18 FedF epitope fusion protein expression, extraction and mouse immunization. Each F18 fimbrial adhesin FedF epitope was genetically fused to human ETEC CFA/I major subunit CfaB as well. PCR amplified epitope fusion genes were digested with NheI and EagI, cloned into expression vector pET28a, and expressed in E. coli BL21. Epitope fusion recombinant proteins were extracted and examined in a standard sodium dodecyl sulfate-polyacrylamide gel electrophoresis (SDS-PAGE) and in ELISA with anti-F18 antiserum (FIGS. 7A and 7B). Coomassie blue staining of SDS-PAGE gel showed all fusion proteins were extracted at the expected size, and Western blot verified recognition with anti-F18 antiserum (FIG. 7A). ELISA confirmed recognition of each fusion protein with anti-F18 antiserum (FIG. 7B).

After protein antigenicity was verified, each FaeG epitope fusion protein was used for mouse immunization. Eight-week old BALB/c mouse (5 mice per group) were each subcutaneously with 40 μg epitope-CfaB fusion protein and 0.2 μg double mutant LT (dmLT) adjuvant. Immunized mice received two boosters (the same dose of the primary) at an interval of two weeks. Two weeks after the second booster injection, mice were euthanized. A group of five mice without immunization was used as the control. Mouse serum samples were collected and stored at −80° C. until use.

Mouse Antibody Titration

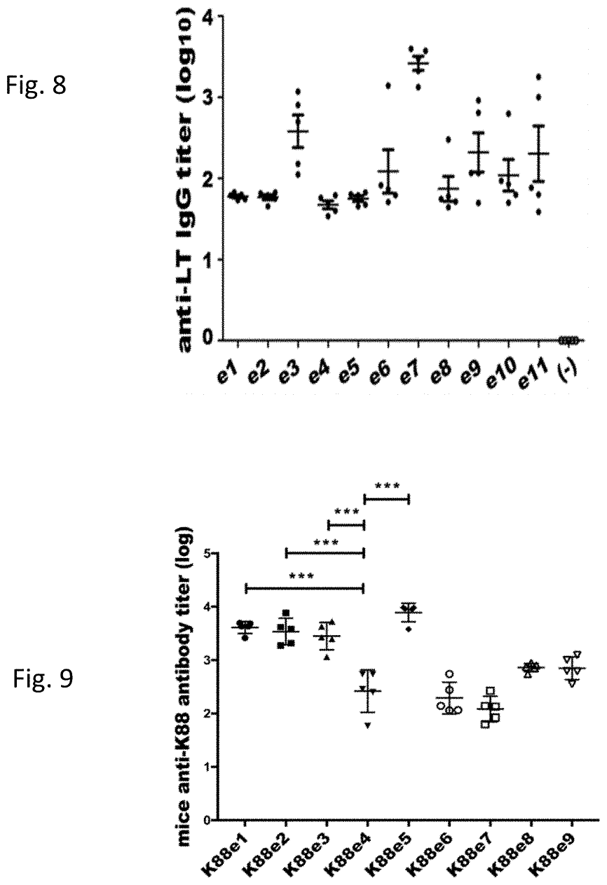

Mice subcutaneously immunized with LT_{Al} epitope fusions developed anti-LT antibody responses. Mice subcutaneously immunized with each epitope-ovalbumin fusion protein developed antibody response to LT (FIG. 8). Mouse serum samples from the group immunized with the fusion protein carrying the e7 epitope developed anti-LT IgG titers significantly greater than titers from other groups. Serum samples of the control groups immunized with ovalbumin protein or PBS had no anti-LT antibody response detected.

Mice subcutaneously immunized with K88 fimbrial subunit FaeG epitope fusions developed anti-K88 antibody responses. All mice subcutaneously immunized with FaeG epitope-CfaB fusion protein developed IgG antibodies to K88 fimbriae (FIG. 9). Mice immunized with fusions carrying epitope 1, 2, 3, or 5 developed significantly greater serum IgG titers. No anti-K88 antibody response was detected in mice immunized with CfaB or PBS.

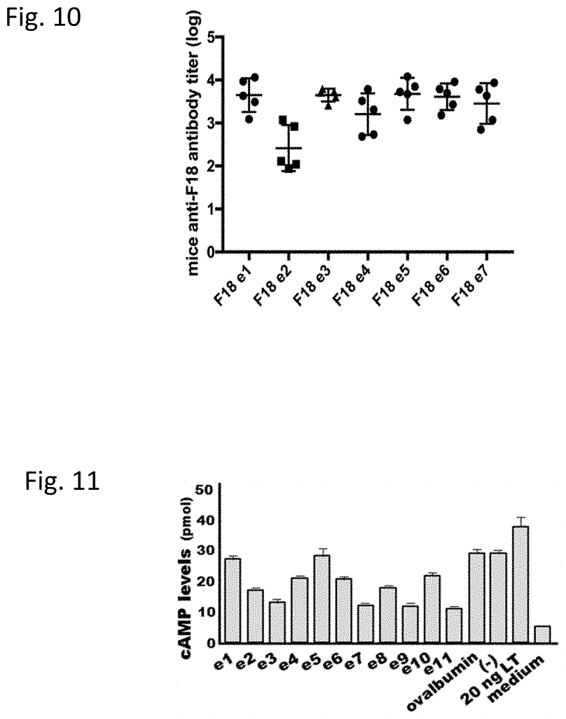

Mice subcutaneously immunized with F18 adhesin subunit FedF epitope fusions developed anti-F18 antibody responses. Each mouse subcutaneously immunized with a F18 FedF epitope-CfaB fusion protein developed anti-F18 IgG antibodies (FIG. 10). No anti-F18 antibody response was detected from the control mouse serum samples.

In vitro Antibody Neutralization Assays

Antibodies derived from LT_{Al} epitope fusion showed neutralizing activities against LT enterotoxicity. Mouse serum samples from the groups immunized with the epitope-ovalbumin fusions except e1-ovalbumin fusion and e5-ovalbumin fusion showed antibody neutralizing activity against LT enterotoxicity (FIG. 11). T-84 cells incubated with 20 ng LT and the serum sample of mice immunized with e2-ovalbumin (17.3±1.7 pmole/ml), e3-ovalbumin (13.7±1.1), e4-ovalbumin (21.7±0.7), e6-ovalbumin (20.9±1.4), e7-ovalbumin (12.2±0.9), e8-ovalbumin (18±0.7), e9-ovalbumin (12.2±0.7), e10-ovalbumin (21.9±1.1), or e11-ovalbumin (11.1±0.7) had significantly lower intracellular cAMP than cells incubated with LT and the serum of the mice immunized with ovalbumin protein (29.3±1.4) or PBS (29.6±1.1). No significantly neutralizing LT activity was detected from the serum samples of the mice immunized with e1-ovalbumin (27.6±1.7; p=0.10) or e5-ovalbumin (28.9±2.2; p=0.60) compared to the serum from the control mice. Mouse serum samples of the groups immunized with e3-, e7-, e9-, or e11-ovlbumin fusion showed significantly greater neutralizing activity against LT compared to the serum samples from the rest immunized groups.

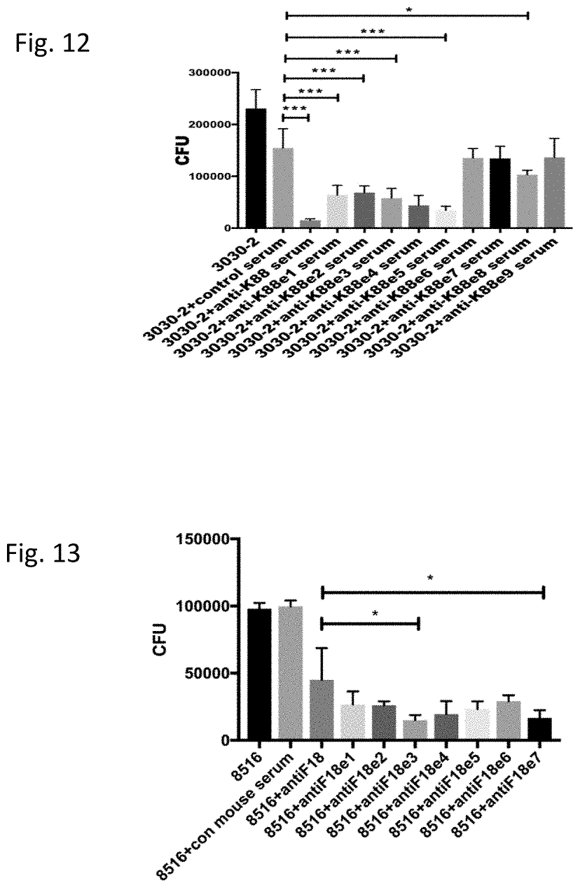

Antibodies derived from K88 FaeG epitope fusion showed antibody in vitro adherence inhibition activities against K88 fimbriae. Mouse serum samples pooled from each immunization group showed inhibition activities against adherence of K88 fimbrial ETEC wild-type strain 30302 to porcine cell line IPEC-J2 cells (FIG. 12). Serum samples from mice immunized with fusion carrying FaeG epitope 1, 2, 3, 4, 5, or 8 showed significant inhibition activity against K88 fimbria adherence, compared to the serum from the control mice. (FIG. 12).

Antibodies derived from F18 FedF epitope fusion showed antibody in vitro adherence inhibition activities against F18 fimbriae. Mouse serum samples pooled from each immunization group showed inhibition activities against adherence of F18 fimbrial ETEC strain 8516 to porcine cell line IPEC-J2 cells (FIG. 13). Serum samples from mice immunized with the fusion protein carrying FedF epitope e3 or e7 showed a significant reduction in bacteria adherent to IPEC-J2 cells.

1. An immunogenic composition comprising a component selected from the group consisting of:
at least one recombinant protein from PEDV or ETEC;
at least one nucleic acid sequence from PEDV inserted into a bacterial strain expressing the inserted nucleic acid sequence;
at least one nucleic acid sequence from ETEC inserted into a vector expressing the inserted nucleic acid sequence; and
any combination thereof.

2. The immunogenic composition of claim 1, wherein the recombinant protein or the nucleic acid sequence is from PEDV and is further selected from the spike protein or the nucleic acid sequence encoding the spike protein of PEDV.

3. The immunogenic composition of claim 1, wherein the recombinant protein is fused into a monomeric heat-labile (LT) toxoid or a holotoxin-structured LT.

4. The immunogenic composition of claim 1, wherein the recombinant protein is expressed as a recombinant protein or as a live strain expressing the holotoxin-structured antigen.

5. The immunogenic composition of claim 4, wherein the live strain expressing the holotoxin-structured antigen is a nonpathogenic E. coli strain.

6. The immunogenic composition of claim 1, wherein the nucleic acid sequence from PEDV is selected from the group consisting of nucleic acid sequences encoding a sequence having at least 80% sequence homology with a sequence selected from the group consisting of SEQ ID NOS. 1-5, or wherein the nucleic acid sequence from ETEC is selected from the group consisting of nucleic acid sequences from the LT_{A} subunit; nucleic acid sequences from the K88 FaeG subunit, nucleic acid sequences from the F18 adhesin subunit; and any combination thereof.

7. (canceled)

8. The immunogenic composition of claim 1, wherein the nucleic acid sequence from ETEC is selected from the group consisting of nucleic acid sequences encoding a sequence having at least 80% sequence homology with a sequence selected from the group consisting of SEQ ID NOS. 9-35, or wherein the recombinant protein from ETEC is selected from the group consisting of SEQ ID NOS. 23, 24, 31, 35, and any combination thereof.

9. (canceled)

10. The immunogenic composition of claim 1, wherein the recombinant ETEC protein or nucleic acid sequence from ETEC is combined with a carrier or backbone.

11. The immunogenic composition of claim 10, wherein the carrier is a modified chicken ovalbumin gene or an E. coli fimbrial subunit gene or any protein expressed thereby.

12. (canceled)

13. (canceled)

14. The immunogenic composition of claim 1, wherein the immunogenic composition is formulated for oral administration.

15. The immunogenic composition of claim 1, further comprising at least one further component selected from the group consisting of an adjuvant, a pharmaceutical-acceptable carrier; a protectant; an immunomodulatory agent, a pharmaceutical acceptable salt at least one immunological active component against another disease-causing organism in swine, and any combination thereof.

16. (canceled)

17. The immunogenic composition of claim 15, wherein the other disease-causing organism in swine is selected from the group consisting of Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl.septicum, Cl. tetani; Coronavirus, preferably Porcine Respiratory Corona virus; Eperythrozoonosis suis; Erysipelothrix rhsiopathiae; Escherichia coil; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa; Leptospira icterohaemorrhagicae; and Leptospira interrogans; Leptospira pomona; Leptospira tarassovi; Mycobacterium spp. preferably M. avium; M. intracellulare; and M. bovis; Mycoplasma hyopneumoniae (M hyo); Pasteurella multocida; Porcine cytomegalovirus; Porcine Parvovirus; Porcine Reproductive and Respiratory Syndrome (PRRS) Virus; Pseudorabies virus; Rotavirus; Salmonella spp.; preferably S. thyhimurium; and S. choleraesuis; Staph. hyicus; Staphylococcus spp. preferably Streptococcus spp., preferably Strep. suis; Swine herpes virus; Swine Influenza Virus; Swine pox virus; Swine pox virus; Vesicular stomatitis virus; Virus of vesicular exanthema of swine; Leptospira Hardjo; Mycoplasma hyosynoviae; and any combination thereof.

18. A method of reducing the incidence of or severity of at least one clinical sign or symptom of infection by PEDV or ETEC comprising the steps of: administering the immunogenic composition of claim 1 to an animal in need thereof.

19. (canceled)

20. The method of claim 18, wherein the administration is oral or via injection.

21. (canceled)

22. The method of claim 18, wherein the administration is repeated.

23. The method of claim 18, wherein the clinical sign is selected from the group consisting of diarrhea, vomiting, anorexia death, dehydration, and any combination thereof for PEDV, or wherein the clinical sign is selected from the group consisting of diarrhea, vomiting, dehydration, roughened hair coat, subnormal body temperature, shivering, death, neonatal septicemia and polyserositis, lesions, excess watery fluid in the small intestine and/or colon, a distended and/or gas-filled small intestine and/or colon, mild reddening and congestion of the stomach, congestion of the gastrointestinal tract, coliforms adhered to microvilli of intestinal epithelial cells, necrosis of villi, microvascular thrombosis in the lamina propria, fibrinous polyserositis, arthritis, and any combination thereof for ETEC.

24. (canceled)

25. The method of claim 18, wherein the immunogenic composition is administered as a recombinant subunit, or as a live vaccine.

26. (canceled)

27. A method of making the immunogenic composition of claim 1, comprising the step of:
inserting a sequence encoding a protein selected from the group consisting of a spike protein of PEDV; the LTA subunit of ETEC; the K88 FaeG subunit of ETEC; the F18 adhesin subunit of ETEC; and any combination thereof into a vector.

28. The method of claim 27, further comprising the step of expressing said protein and/or of combining the expressed protein with a pharmaceutically acceptable carrier.

29. (canceled)

30. (canceled)

31. A kit comprising a component selected from the group consisting of:
at least one recombinant protein from PEDV or ETEC;
at least one nucleic acid sequence from PEDV inserted into a bacterial strain expressing the inserted nucleic acid sequence;
at least one nucleic acid sequence from ETEC inserted into a vector expressing the inserted nucleic acid sequence; and
any combination thereof; and
a set of instructions for administration to an animal in need thereof.


SEQUENCE LISTING (35 sequences)

SEQ ID NO: 1 - protein, 13 aa, porcine epidemic diarrhea virus
HGKVVSNQPLLVN

SEQ ID NO: 2 - protein, 13 aa, porcine epidemic diarrhea virus
GPRLQPYEVFEKV

SEQ ID NO: 3 - protein, 8 aa, porcine epidemic diarrhea virus
YSNIGVCK

SEQ ID NO: 4 - protein, 8 aa, porcine epidemic diarrhea virus
SQSGQVKI

SEQ ID NO: 5 - protein, 19 aa, porcine epidemic diarrhea virus
PYYCFFKVDTYNSTVYKFL

SEQ ID NO: 6 - protein, 5 aa, Artificial Sequence
  Linker sequence
GGGGS

SEQ ID NO: 7 - protein, 5 aa, Artificial Sequence
  Linker sequence
GPGPG

SEQ ID NO: 8 - DNA, 1237 bp, porcine epidemic diarrhea virus
ttgacatcatgttgcatataggttagataaaacaagtggcgttatctttttccggattgtcttcttgtatgatatataagttttcctcgatgaaaaatataactttcattttttttattttattagcatcgccattatatgcaaatggcgacagattataccgtgctgactctagacccccagatgaaataaaacgttccggaggtcttatgcccagaggaggtggaggaagctatagtaacataggtgtttgtaaaattaatctttatgatcatggtaaggtggtttccaaccaaccattgttggtcaatgacggatatgtttctcagtctggccaagtcaagattgctcacttagcaggacagtctatattatcaggatattccacttactatatatatgttatagcgacagcaccaaatatgtttaatgttaatgatgtattaggcgtatacggtcctagacttcaaccttacgaagtttttgaaaaggtcggaataccatattctcagatatatggatggtatcgtgttaattttggtccctattattgtttttttaaagtggatacttacaactccactgtttataaattcttaggacccggacctggtgcagaagatggttacagattagcaggtttcccaccggatcaccaagcttggagagaagaaccctggattcatcatgcaccacaaggttgtggaaattcatcaagaacaatcacaggtgatacttgtaatgaggagacccagaatctgagcacaatatatctcagggaatatcaatcaaaagttaagaggcagatattttcagactatcagtcagaggttgacatatataacagaattcgggatgaattatgaataaagtaaaatgttatgttttatttacggcgttactatcctctctatatgcacacggagctccccagactattacagaactatgttcggaatatcgcaacacacaaatatatacgataaatgacaagatactatcatatacggaatcgatggcaggcaaaagagaaatggttatcattacatttaagagcggcgaaacatttcaggtcgaagtcccgggcagtcaacatatagactcccagaaaaaagccattgaaaggatgaaggacacattaagaatcacatatctgaccgagaccaaaattgataaattatgtgtatggaataataaaacccccaattcaattgcggcaatcagtatgaaaaactag

SEQ ID NO: 9 - protein, 12 aa, Escherichia coli
TITGDTCNEETQ

SEQ ID NO: 10 - protein, 11 aa, Escherichia coli
NGDKLYRADSR

SEQ ID NO: 11 - protein, 13 aa, Escherichia coli
DSRPPDEIKRSGG

SEQ ID NO: 12 - protein, 12 aa, Escherichia coli
RGHNEYFDRGTQ

SEQ ID NO: 13 - protein, 10 aa, Escherichia coli
YDHARGTQTG

SEQ ID NO: 14 - protein, 10 aa, Escherichia coli
RYDDGYVSTS

SEQ ID NO: 15 - protein, 11 aa, Escherichia coli
SPHPYEQEVSA

SEQ ID NO: 16 - protein, 10 aa, Escherichia coli
HRNREYRDRY

SEQ ID NO: 17 - protein, 11 aa, Escherichia coli
APAEDGYRLAG

SEQ ID NO: 18 - protein, 13 aa, Escherichia coli
AGFPPDHQAWREE

SEQ ID NO: 19 - protein, 13 aa, Escherichia coli
HHAPQGCGNSSRT

SEQ ID NO: 20 - protein, 10 aa, Escherichia coli
MTGDFNGSVD

SEQ ID NO: 21 - protein, 10 aa, Escherichia coli
LNDLTNGGTK

SEQ ID NO: 22 - protein, 10 aa, Escherichia coli
GRTKEAFATP

SEQ ID NO: 23 - protein, 10 aa, Escherichia coli
ELRKPDGGTN

SEQ ID NO: 24 - protein, 16 aa, Escherichia coli
PMKNAGGTKVGSVKVN

SEQ ID NO: 25 - protein, 12 aa, Escherichia coli
GRGGVTSADGEL

SEQ ID NO: 26 - protein, 11 aa, Escherichia coli
PRGSELSAGSA

SEQ ID NO: 27 - protein, 10 aa, Escherichia coli
RENMEYTDGT

SEQ ID NO: 28 - protein, 10 aa, Escherichia coli
FNQAVTTSTQ

SEQ ID NO: 29 - protein, 10 aa, Escherichia coli
INSSASSAQV

SEQ ID NO: 30 - protein, 11 aa, Escherichia coli
LGTGKTNTTQM

SEQ ID NO: 31 - protein, 14 aa, Escherichia coli
IPSSSGTLTCQAGT

SEQ ID NO: 32 - protein, 10 aa, Escherichia coli
NESQWGQQSQ

SEQ ID NO: 33 - protein, 10 aa, Escherichia coli
AQTYPLSSGD

SEQ ID NO: 34 - protein, 10 aa, Escherichia coli
PNQNDMPSSN

SEQ ID NO: 35 - protein, 10 aa, Escherichia coli
QPDATGSWYD